SEQUENCE LISTING (58 sequences)

SEQ ID NO: 1 - DNA, 15489 bp, synthetic construct
  source 1..15489: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccctaattagctaaggtaccttatagccgtttctttgttttttttcttctgttgagctccaactttatgaaacgtcgtaagctggtaattatgaaatgtaaggatttggagagagaaaaaaaacgggagggaaaaccatgcatgctgctgacgcgacggccggacgcagacgcaacaatgcccccggtgcggcgttgtcgagcagccactgcaccaccccacgcatcacctgcagtaatctagcgacgggtttttcttatttatttatttatttatttatttttcttctctccctccctccctcagatttgttttcgttttcattaatcgttattaccagcaattaattaactttatctattgatttaccaaaccgcaataaagaatatatatattcttttattaaggtccagtaataagcagcacagaagcgcaggtgcagcagcagcagcgtcagcgcccgaggcgcgcacgagagaaacagaggctgacgaggtggggcccgtgcgggccttgaccaatcggagttcgacaacagcctggccacccacaaacacacactccttcgcctcgcgccggccgtcgtcgcctccctccaccgaacgatccctcctcctcctcctcctcctcctcctcgcatcccaccccaccccaccttctccttaaagctacctgcctacccggcggttgccgccgccgcaatcgatcgaccggaagagaaagagcagctagctagctagcagatcggagcacggcaacaaggcgaaggggcgcggcaaggtacagctgaagcggatagagaacaagataaaccggcaggtgaccttctccaagcgccggaacggcctgctcaagaaggcgcacgagatctccgtcctctgcgttgccgaggtcgccgtcatcgtcttctcccccaagggcaagctctacgagtacgccaccgactcccggtacgtacgaccaccttcaattcgtcacacaggcagtgtgtttgttgaattgaatacgtgcctgctatgctagctgcctgtcagtatggtccatccgttcgccatcagttcatctttctgcttcgtgctgctgttgtgtgtgtgcgccataccatgccatgccatgccatgccaccaacctggctgtgctgccgtttttggctcgcagccgccgatccccgacccgaaagctcatgctgctcttgccgttttcggccaccacccaccatggcctgctgtctggtcctccacgcctgcttccccagttccacttccatgcatggtggtcagttggctggcatggcagcagcagccgccttctggttcgtttgtgctgcctcaggatttgctttctactagctagtgtgctacagccgatcatttcatgctgctgcccgcgtcgatctgctccgctagctgcttcctcgtttcgggaaacctcgcccgccccgccattaactgattcacctgctttccttctgttgcggtgttgcctacgacgactgaaacctttcctttttggggattggcgtctgccgactccactaggcctccttttaattccttgttttatatacatgtacataagcttttgctgatgcagtggtacggtgcctttatttttatcggattacagtacgtgttagattgataacagcacgcaatcagatctagctttgcttgcttccttccttcctcgtcaaatctttctggcccttctcgatttgcgctccatcgtataattaattaaccatcactaatcacactttccccatttttctaaacaaaaaaaagtatcacatgttcctaatcacgcgtgcatgtaatttgctgtttctttctctctctctctcaattaatttgctgcctgccgagaaacacgagctgcatgctggctcaatcagttcctaactgcgaaagtgcagataatgaccccctcggctcggattaatcagtacacatgatcgaggacgcatagatctggcttttccttatctggttcttcgtcttcaattctccgtttcttccctcgcatccttttccgtgttgtttcagatttacaaataaaccatcgtctctctcactttcatctgttgttcttccatgacagtgaaaaatactgttttttcctcgggtgcatggaaaaattaaaattaaaggcccgtgtttttttctctttggatgtgttcttataggatggatacactatgtgcatccgggttttgtggtgatgtaacatagtaaagcagctccgaggtactgggttctatttcatattccccccccccccaaatcaaaaaagttatctttgctcgaaggtctactcgtttttttttctggcttttatacgaattctatgacatgctagtatgggtaaaaacgaaatggtgattctcattcgtatatgcactattatattctttgcatgcacttttattacaaaagctgcatggacgtgttacacattcacggagataacaattgagcagtagattagtttctgtcttcttaattagctaggcaggctcagaggtgaaactgcagctattaggttgagtaacacagctagtgctgtattcatgaagcttcggtttgactcagttttgcagtagcactggttgcaactcgctaaaccatttaacttccatgtacatgatgtctttgtaggagtttgtgttcattcttcttattttgacatttcaagtcattcagtacgtaactactaaaataagtgtcttgcagagtcagtctcaagataacttgttggtcttggcttattagctaagatgcaatgcatcattacttgccttactgtgatggctaaaccaatatcacatgcactgtgtgctttttcaagcattccacatccatacatacctagtatccctgactttttaatgacccctgatgatagaacatgcattgtttgtatattacctgttagtgttcttaacataaaattttacaaaagaagctctattacgaggatgttatgctgcttttgcaaaggaaactagctaggcagactggtgctttttaaataaacaattaaaatcttgtgaacccaaacactacttacagatcgaaaattacagtaagacgaaagtaaatggcaactagtacaatactcgtacatgcgttgcgacggcacataaaaacaaatattacgttttatcttagccaaagcgtctagaaaggtatgagttgaaaagaagactttttatagcttactcggtcgctgttcttcctttagctagcaagttaatactatatgggagcgctacgcagtgtgccgcttcctctcgtgttgggctcaggtggtttctcacagtccatctgtaaggtaacagcacactaataggagcttcagtgggcttatttatgagataacagcctactcattaatacacctacatgtgaaaggacgatcgtcgaaactggtgttttatagtagtagaaatgaactttgaggcatagcttctttgcaattccattgttagcttgtttttttgttttgaagatacatgtcgttagcttacttgttggattgcatgtaaacatgcatcatggttgctttagaaaattttgttgacgttgatgagtacatttctctatcgcagtacaagatatgtaatgatatagtacttgcatgctcgtgctttcttgcatatgattgcttttcgaaagggaatataaatgccctcgaaaatttacttatcccttgcaatgatctctggtttaagcatagattatttcaaggacttccaaagaaaatgatttgacattcttttttccccttttttcagcggatcctaccctgcgcctgcgcgtccaccggtggactcgtcgacaccatcatcgaaggcaagaccgggttccacatcggccgcctcagcgtcgacgtaacgtcgtggagccggcggacgtcaagaaggtggccaccacattgcagcgcgccttcaaggtggtcggcacgccggcgtacgaggagatggtgaggaactgcttgatccaggatctctcctggaagggccctgccaagaactgggagaacgtgctgctcagcctcggggtcgccggcggctagccaggggtcgaaggcgaggagatcgcgccgctcgccaaggagaacgtggccgcgccctaattagctagtcgacgtaagcgcgcgcaccgagacatgcatccgttggatcgcgtcttcttcgtgctcttgccgcgtgcatgatgcatgtgtttcctcctggcttgtgttcgtgtatgtgacgtgtttgttcgggcatgcatgcagtcgactagtcagggcgcggccacgttctccttggcgagcggcgcgatctcctcgccttcgacccctggctagccgccggcgaccccgaggctgagcagcacgttctcccagttcttggcagggcccttccaggagagatcctggatcaagcagttcctcaccatctcctcgtacgccggcgtgccgaccaccttgaaggcgcgctgcaatgtggtggccaccttcttgacgtccgccggctccacgacgttacgtcgacgctgaggcggccgatgtggaacccggtcttgccttcgatgatggtgtcgacgagtccaccggtggacgcgcaggcgcagggcctaggagctcaagctacggagagggtcgatggatcatgacgtacgcgtaccatatggttgctgtgcctgcccccatcgatcgcgagcaatggcacgctcatgcaagtgatcattgctccctgttggttaaaccctagcctatgttcatggcgtcagcaactaagctaaactattgttatgtttgcaagaaagggtaaacccgctagctgtgtaatcttgtccagctaccatgtatgcttgttactgcccagttacccttgaaatctaggggcgcttttggtgagagggtgcagtttactttaaacatggttcgtgacttgctgtaaatagtagtattaatcgatttgggcatctagtaagtaaattaagggtgtgtttgatttggcttttactctctaaaagttaaaagctaaattaaagagctggatctagaaaatagacttttctaaaagccgactttgtgatggtgcaaaattgaaagcgtcttagcttttggctaaagagttgtttggtttgactcttttgtcctctaaaaaccaaaagtcaaaccaaagagctagatctaaaaacacgtttttttttctttctaaaaatcgtctttctcatttggacctacttttagtgtcttttaaagcatctttggacctactttttacggcttttaatggtttcactaaatggtttttaactttttaacaactcacaatctacatcaatttttttttcatagcttgtagtctacaacaacattttttacaacctacaatctaaccaaatagaccctaactaatgggagaatgaaaaaaaaatcgctcgatcttatgctattgatgcatgacatgtgaaaccatgaggctcatttttgaacgattggatacataaacctagcgatgcttcactgtgacatccggcgcatatctgtatgtttagtgtactttgtttctgtagtttttccttaagtgtagtgcaacttcttatctggtgttttattcggtgtatcgttgtaatcatctagtacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatctccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 2 - DNA, 18160 bp, synthetic construct
  source 1..18160: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcccctaaatagaccatgccgaagaagaagcgcaaggtcgggggcgggggctcaggcgggggcgggagcggcggcgggggctctgggggcggcggcagcggcgggggcggcagcgggggcggcgggtcgatgagcaagctggagaagttcacgaactgctactccctcagcaagaccctgaggttcaaggcgatcccggtcggcaagacccaggagaacatcgacaacaagcggctgctggtggaggacgagaagagggctgaggactacaagggcgtgaagaagctcctggaccgctactacctgtccttcatcaacgacgtgctccacagcatcaagctcaagaacctgaacaactacatcagcctcttcaggaagaagacgcgcaccgagaaggagaacaaggagctcgagaacctggagatcaacctgaggaaggagatcgccaaggcgttcaagggcaacgagggctacaagtccctcttcaagaaggacatcatcgagacgatcctcccggagttcctggacgacaaggacgagatcgccctggtcaactccttcaacggcttcaccacggcgttcaccggcttcttcgacaaccgcgagaacatgttcagcgaggaggccaagtccacgagcatcgcgttcaggtgcatcaacgagaacctcacccgctacatctccaacatggacatcttcgagaaggtcgacgcgatcttcgacaagcacgaggtgcaggagatcaaggagaagatcctgaacagcgactacgacgtcgaggacttcttcgagggcgagttcttcaacttcgtcctcacgcaggagggcatcgacgtgtacaacgccatcatcggtggcttcgtgaccgagtccggcgagaagatcaagggcctgaacgagtacatcaacctctacaaccagaagaccaagcagaagctgccgaagttcaagcccctgtacaagcaggtgctctccgacagggagtccctcagcttctacggcgagggctacacgagcgacgaggaggtcctggaggtgttccgcaacaccctcaacaagaacagcgagatcttctccagcatcaagaagctcgagaagctgttcaagaacttcgacgagtactccagcgccggcatcttcgtcaagaacggcccggcgatctccacgatcagcaaggacatcttcggcgagtggaacgtgatccgcgacaagtggaacgccgagtacgacgacatccacctcaagaagaaggcggtggtcaccgagaagtacgaggacgacaggcgcaagtccttcaagaagatcggctccttcagcctcgagcagctgcaggagtacgccgacgcggacctgagcgtggtcgagaagctcaaggagatcatcatccagaaggtcgacgagatctacaaggtgtacggctccagcgagaagctcttcgacgcggacttcgtcctcgagaagtccctgaagaagaacgacgccgtggtcgcgatcatgaaggacctcctggactccgtgaagagcttcgagaattacatcaaggccttcttcggcgagggcaaggagacgaacagggacgagtccttctacggcgacttcgtcctggcctacgacatcctcctgaaggtggaccacatctacgacgcgatccgcaactacgtgacccagaagccgtacagcaaggacaagttcaagctctacttccagaacccccagttcatgggcggctgggacaaggacaaggagacggactacagggcgaccatcctgcgctacggcagcaagtactacctcgccatcatggacaagaagtacgcgaagtgcctgcagaagatcgacaaggacgacgtcaacggcaactacgagaagatcaactacaagctcctgccgggccccaacaagatgctcccgaaggtgttcttctccaagaagtggatggcctactacaaccccagcgaggacatccagaagatctacaagaacggcacgttcaagaagggcgacatgttcaacctgaacgactgccacaagctcatcgacttcttcaaggactccatcagccgctacccgaagtggtccaacgcctacgacttcaacttcagcgagaccgagaagtacaaggacatcgcgggcttctaccgcgaggtcgaggagcagggctacaaggtgtccttcgagtccgccagcaagaaggaggtcgacaagctggtggaggagggcaagctctacatgttccagatctacaacaaggacttctccgacaagagccacggcacgcccaacctgcacaccatgtacttcaagctcctgttcgacgagaacaaccacggccagatcaggctgtccggcggcgccgagctcttcatgaggagggcgagcctgaagaaggaggagctggtggtccaccccgctaacagcccaatcgcgaacaagaacccggacaaccccaagaagaccacgaccctgtcctacgacgtgtacaaggacaagaggttcagcgaggaccagtacgagctccacatcccgatcgcgatcaacaagtgccccaagaacatcttcaagatcaacaccgaggtccgcgtgctcctgaagcacgacgacaacccctacgtgatcggcatcgacaggggcgagaggaacctcctgtacatcgtggtcgtggacggcaagggcaacatcgtggagcagtactccctcaacgagatcatcaacaacttcaacggcatcaggatcaagacggactaccacagcctcctggacaagaaggagaaggagaggttcgaggcccgccagaactggacctccatcgagaacatcaaggagctgaaggcgggctacatcagccaggtcgtgcacaagatctgcgagctcgtcgagaagtacgacgccgtgatcgccctcgaggacctgaactccggcttcaagaacagccgcgtcaaggtggagaagcaggtctaccagaagttcgagaagatgctcatcgacaagctgaactacatggtggacaagaagtccaacccctgcgctacgggcggcgcgctgaagggctaccagatcaccaacaagttcgagagcttcaagtccatgagcactcagaacggcttcatcttctacatcccggcgtggctcacgtccaagatcgaccccagcaccggcttcgtcaacctcctgaagacgaagtacacctccatcgccgacagcaagaagttcatctccagcttcgaccgcatcatgtatgtgccggaggaggacctgttcgagttcgccctcgactacaagaacttctcccgcacggacgcggactacatcaagaagtggaagctgtacagctacggcaaccgcatccgcatcttcaggaaccccaagaagaacaacgtcttcgactgggaggaggtgtgcctgacctccgcgtacaaggagctcttcaacaagtacggcatcaactaccagcagggcgacatcagggctctcctgtgcgagcagagcgacaaggccttctactccagcttcatggcgctgatgtccctcatgctgcagatgaggaactcgatcaccggcaggacggacgtggacttcctcatctccccggtgaagaacagcgacggcatcttctacgactccaggaactacgaggcccaggagaacgcgatcctcccaaagaacgcggacgccaacggcgcctacaacatcgccaggaaggtcctctgggctatcggccagttcaagaaggcggaggacgagaagctggacaaggtgaagatcgccatcagcaacaaggagtggctcgagtacgcccagacctcggtcaagcacggcagcccgaagaagaagcgcaaggtgtccggcggcagctccggcggcagcccgaagaagaagcgcaaagtgtgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaacgcgttaactagctaggaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggtgcgcaggtacctaaattactgatgagtccgtgaggacgaaacgagtaagctcgtctaatttctactaagtgtagatagcaggatcctccggacaatttcggccggcatggtcccagcctcctcgctggcgccggctgggcaacatgcttcggcatggcgaatgggacgaagaaattactgatgagtccgtgaggacgaaacgagtaagctcgtctaatttctactaagtgtagatcccactccatgttgattacctgtggccggcatggtcccagcctcctcgctggcgccggctgggcaacatgcttcggcatggcgaatgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccggaccgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagctaaaccatgcagaagctgatcaacagcgtgcagaactacgcctggggcagcaagaccgccctgaccgagctgtacggcatggagaaccccagcagccagccaatggccgagctgtggatgggcgcccaccccaaaagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatcccaatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagactccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagactactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgataggattacctagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctgctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgatgagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 3 - DNA, 15604 bp, synthetic construct
  source 1..15604: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccctaattagctaaggtaccttatagccgtttctttgttttttttcttctgttgagctccaactttatgaaacgtcgtaagctggtaattatgaaatgtaaggatttggagagagaaaaaaaacgggagggaaaaccatgcatgctgctgacgcgacggccggacgcagacgcaacaatgcccccggtgcggcgttgtcgagcagccactgcaccaccccacgcatcacctgcagtaatctagcgacgggtttttcttatttatttatttatttatttatttttcttctctccctccctccctcagatttgttttcgttttcattaatcgttattaccagcaattaattaactttatctattgatttaccaaaccgcaataaagaatatatatattcttttattaaggtccagtaataagcagcacagaagcgcaggtgcagcagcagcagcgtcagcgcccgaggcgcgcacgagagaaacagaggctgacgaggtggggcccgtgcgggccttgaccaatcggagttcgacaacagcctggccacccacaaacacacactccttcgcctcgcgccggccgtcgtcgcctccctccaccgaacgatccctcctcctcctcctcctcctcctcctcgcatcccaccccaccccaccttctccttaaagctacctgcctacccggcggttgccgccgccgcaatcgatcgaccggaagagaaagagcagctagctagctagcagatcggagcacggcaacaaggcgaaggggcgcggcaaggtacagctgaagcggatagagaacaagataaaccggcaggtgaccttctccaagcgccggaacggcctgctcaagaaggcgcacgagatctccgtcctctgcgttgccgaggtcgccgtcatcgtcttctcccccaagggcaagctctacgagtacgccaccgactcccggtacgtacgaccaccttcaattcgtcacacaggcagtgtgtttgttgaattgaatacgtgcctgctatgctagctgcctgtcagtatggtccatccgttcgccatcagttcatctttctgcttcgtgctgctgttgtgtgtgtgcgccataccatgccatgccatgccatgccaccaacctggctgtgctgccgtttttggctcgcagccgccgatccccgacccgaaagctcatgctgctcttgccgttttcggccaccacccaccatggcctgctgtctggtcctccacgcctgcttccccagttccacttccatgcatggtggtcagttggctggcatggcagcagcagccgccttctggttcgtttgtgctgcctcaggatttgctttctactagctagtgtgctacagccgatcatttcatgctgctgcccgcgtcgatctgctccgctagctgcttcctcgtttcgggaaacctcgcccgccccgccattaactgattcacctgctttccttctgttgcggtgttgcctacgacgactgaaacctttcctttttggggattggcgtctgccgactccactaggcctccttttaattccttgttttatatacatgtacataagcttttgctgatgcagtggtacggtgcctttatttttatcggattacagtacgtgttagattgataacagcacgcaatcagatctagctttgcttgcttccttccttcctcgtcaaatctttctggcccttctcgatttgcgctccatcgtataattaattaaccatcactaatcacactttccccatttttctaaacaaaaaaaagtatcacatgttcctaatcacgcgtgcatgtaatttgctgtttctttctctctctctctcaattaatttgctgcctgccgagaaacacgagctgcatgctggctcaatcagttcctaactgcgaaagtgcagataatgaccccctcggctcggattaatcagtacacatgatcgaggacgcatagatctggcttttccttatctggttcttcgtcttcaattctccgtttcttccctcgcatccttttccgtgttgtttcagatttacaaataaaccatcgtctctctcactttcatctgttgttcttccatgacagtgaaaaatactgttttttcctcgggtgcatggaaaaattaaaattaaaggcccgtgtttttttctctttggatgtgttcttataggatggatacactatgtgcatccgggttttgtggtgatgtaacatagtaaagcagctccgaggtactgggttctatttcatattccccccccccccaaatcaaaaaagttatctttgctcgaaggtctactcgtttttttttctggcttttatacgaattctatgacatgctagtatgggtaaaaacgaaatggtgattctcattcgtatatgcactattatattctttgcatgcacttttattacaaaagctgcatggacgtgttacacattcacggagataacaattgagcagtagattagtttctgtcttcttaattagctaggcaggctcagaggtgaaactgcagctattaggttgagtaacacagctagtgctgtattcatgaagcttcggtttgactcagttttgcagtagcactggttgcaactcgctaaaccatttaacttccatgtacatgatgtctttgtaggagtttgtgttcattcttcttattttgacatttcaagtcattcagtacgtaactactaaaataagtgtcttgcagagtcagtctcaagataacttgttggtcttggcttattagctaagatgcaatgcatcattacttgccttactgtgatggctaaaccaatatcacatgcactgtgtgctttttcaagcattccacatccatacatacctagtatccctgactttttaatgacccctgatgatagaacatgcattgtttgtatattacctgttagtgttcttaacataaaattttacaaaagaagctctattacgaggatgttatgctgcttttgcaaaggaaactagctaggcagactggtgctttttaaataaacaattaaaatcttgtgaacccaaacactacttacagatcgaaaattacagtaagacgaaagtaaatggcaactagtacaatactcgtacatgcgttgcgacggcacataaaaacaaatattacgttttatcttagccaaagcgtctagaaaggtatgagttgaaaagaagactttttatagcttactcggtcgctgttcttcctttagctagcaagttaatactatatgggagcgctacgcagtgtgccgcttcctctcgtgttgggctcaggtggtttctcacagtccatctgtaaggtaacagcacactaataggagcttcagtgggcttatttatgagataacagcctactcattaatacacctacatgtgaaaggacgatcgtcgaaactggtgttttatagtagtagaaatgaactttgaggcatagcttctttgcaattccattgttagcttgtttttttgttttgaagatacatgtcgttagcttacttgttggattgcatgtaaacatgcatcatggttgctttagaaaattttgttgacgttgatgagtacatttctctatcgcagtacaagatatgtaatgatatagtacttgcatgctcgtgctttcttgcatatgattgcttttcgaaagggaatataaatgccctcgaaaatttacttatcccttgcaatgatctctggtttaagcatagattatttcaaggacttccaaagaaaatgatttgacattcttttttccccttttttcagcggatccggatgaacatggtctgtttaatgcactggaccttggcgggaccaactttcgtgtcatccgggtccaacttggcggaagggacagacgtgtcgtgaagctacagtatgaagaggtctccattccgcctcatcttatggttggaacttctacggaactatttgatttcattgctgctgagttggaacaatttgtgcggactgaaggagaagatttccacctaccagatggcaagcagagggaactgggtttcaccttttctttcccagtgcaccaaccatctatatcatcggggactctaatctagtcgacgttagtgtttaacttccatcgagtgagtggaattttaatagtctttggaggatgaactagctcaattgatgcgaagtgtggttgtgcggcccaccactcaatgcaaattcccttcaactcgaatttgggttcatatttcttcttagttacaatgccacctagttcctactatgttggtcaagtttttattttaaatgatcctttgcaccttgctaaattgtatccgtccaacataagtttgccttctgtaattagtatcttaatatttctcaccagttaacaatttaaaatttggttcttttgaaatgctacagtcgactagtcattagagtccccgatgatatagatggttggtgcactgggaaagaaaaggtgaaacccagttccctctgcttgccatctggtaggtggaaatcttctccttcagtccgcacaaattgttccaactcagcagcaatgaaatcaaatagttccgtagaagttccaaccataagatgaggcggaatggagacctcttcatactgtagcttcacgacacgtctgtcccttccgccaagttggacccggatgacacgaaagttggtcccgccaaggtccagtgcattaaacagaccatgttcatcccctaggagctcaagctacggagagggtcgatggatcatgacgtacgcgtaccatatggttgctgtgcctgcccccatcgatcgcgagcaatggcacgctcatgcaagtgatcattgctccctgttggttaaaccctagcctatgttcatggcgtcagcaactaagctaaactattgttatgtttgcaagaaagggtaaacccgctagctgtgtaatcttgtccagctaccatgtatgcttgttactgcccagttacccttgaaatctaggggcgcttttggtgagagggtgcagtttactttaaacatggttcgtgacttgctgtaaatagtagtattaatcgatttgggcatctagtaagtaaattaagggtgtgtttgatttggcttttactctctaaaagttaaaagctaaattaaagagctggatctagaaaatagacttttctaaaagccgactttgtgatggtgcaaaattgaaagcgtcttagcttttggctaaagagttgtttggtttgactcttttgtcctctaaaaaccaaaagtcaaaccaaagagctagatctaaaaacacgtttttttttctttctaaaaatcgtctttctcatttggacctacttttagtgtcttttaaagcatctttggacctactttttacggcttttaatggtttcactaaatggtttttaactttttaacaactcacaatctacatcaatttttttttcatagcttgtagtctacaacaacattttttacaacctacaatctaaccaaatagaccctaactaatgggagaatgaaaaaaaaatcgctcgatcttatgctattgatgcatgacatgtgaaaccatgaggctcatttttgaacgattggatacataaacctagcgatgcttcactgtgacatccggcgcatatctgtatgtttagtgtactttgtttctgtagtttttccttaagtgtagtgcaacttcttatctggtgttttattcggtgtatcgttgtaatcatctagtacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatctccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 4 - DNA, 22233 bp, synthetic construct
  source 1..22233: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccctaaaggtaccttatagccgtttctttgttttttttcttctgttgagctccaactttatgaaacgtcgtaagctggtaattatgaaatgtaaggatttggagagagaaaaaaaacgggagggaaaaccatgcatgctgctgacgcgacggccggacgcagacgcaacaatgcccccggtgcggcgttgtcgagcagccactgcaccaccccacgcatcacctgcagtaatctagcgacgggtttttcttatttatttatttatttatttatttttcttctctccctccctccctcagatttgttttcgttttcattaatcgttattaccagcaattaattaactttatctattgatttaccaaaccgcaataaagaatatatatattcttttattaaggtccagtaataagcagcacagaagcgcaggtgcagcagcagcagcgtcagcgcccgaggcgcgcacgagagaaacagaggctgacgaggtggggcccgtgcgggccttgaccaatcggagttcgacaacagcctggccacccacaaacacacactccttcgcctcgcgccggccgtcgtcgcctccctccaccgaacgatccctcctcctcctcctcctcctcctcctcgcatcccaccccaccccaccttctccttaaagctacctgcctacccggcggttgccgccgccgcaatcgatcgaccggaagagaaagagcagctagctagctagcagatcggagcacggcaacaaggcgaaggggcgcggcaaggtacagctgaagcggatagagaacaagataaaccggcaggtgaccttctccaagcgccggaacggcctgctcaagaaggcgcacgagatctccgtcctctgcgttgccgaggtcgccgtcatcgtcttctcccccaagggcaagctctacgagtacgccaccgactcccggtacgtacgaccaccttcaattcgtcacacaggcagtgtgtttgttgaattgaatacgtgcctgctatgctagctgcctgtcagtatggtccatccgttcgccatcagttcatctttctgcttcgtgctgctgttgtgtgtgtgcgccataccatgccatgccatgccatgccaccaacctggctgtgctgccgtttttggctcgcagccgccgatccccgacccgaaagctcatgctgctcttgccgttttcggccaccacccaccatggcctgctgtctggtcctccacgcctgcttccccagttccacttccatgcatggtggtcagttggctggcatggcagcagcagccgccttctggttcgtttgtgctgcctcaggatttgctttctactagctagtgtgctacagccgatcatttcatgctgctgcccgcgtcgatctgctccgctagctgcttcctcgtttcgggaaacctcgcccgccccgccattaactgattcacctgctttccttctgttgcggtgttgcctacgacgactgaaacctttcctttttggggattggcgtctgccgactccactaggcctccttttaattccttgttttatatacatgtacataagcttttgctgatgcagtggtacggtgcctttatttttatcggattacagtacgtgttagattgataacagcacgcaatcagatctagctttgcttgcttccttccttcctcgtcaaatctttctggcccttctcgatttgcgctccatcgtataattaattaaccatcactaatcacactttccccatttttctaaacaaaaaaaagtatcacatgttcctaatcacgcgtgcatgtaatttgctgtttctttctctctctctctcaattaatttgctgcctgccgagaaacacgagctgcatgctggctcaatcagttcctaactgcgaaagtgcagataatgaccccctcggctcggattaatcagtacacatgatcgaggacgcatagatctggcttttccttatctggttcttcgtcttcaattctccgtttcttccctcgcatccttttccgtgttgtttcagatttacaaataaaccatcgtctgtctcactttcatctgttgttcttccatgacagtgaaaaatactgttttttcctcgggtgcatggaaaaattaaaattaaaggcccgtgtttttttctctttggatgtgttcttataggatggatacactatgtgcatccgggttttgtggtgatgtaacatagtaaagcagctccgaggtactgggttctatttcatattccccccccccccaaatcaaaaaagttatctttgctcgaaggtctactcgtttttttttctggcttttatacgaattctatgacatgctagtatgggtaaaaacgaaatggtgattctcattcgtatatgcactattatattctttgcatgcacttttattacaaaagctgcatggacgtgttacacattcacggagataacaattgagcagtagattagtttctgtcttcttaattagctaggcaggctcagaggtgaaactgcagctattaggttgagtaacacagctagtgctgtattcatgaagcttcggtttgactcagttttgcagtagcactggttgcaactcgctaaaccatttaacttccatgtacatgatgtctttgtaggagtttgtgttcattcttcttattttgacatttcaagtcattcagtacgtaactactaaaataagtgtcttgcagagtcagtctcaagataacttgttggtcttggcttattagctaagatgcaatgcatcattacttgccttactgtgatggctaaaccaatatcacatgcactgtgtgctttttcaagcattccacatccatacatacctagtatccctgactttttaatgacccctgatgatagaacatgcattgtttgtatattacctgttagtgttcttaacataaaattttacaaaagaagctctattacgaggatgttatgctgcttttgcaaaggaaactagctaggcagactggtgctttttaaataaacaattaaaatcttgtgaacccaaacactacttacagatcgaaaattacagtaagacgaaagtaaatggcaactagtacaatactcgtacatgcgttgcgacggcacataaaaacaaatattacgttttatcttagccaaagcgtctagaaaggtatgagttgaaaagaagactttttatagcttactcggtcgctgttcttcctttagctagcaagttaatactatatgggagcgctacgcagtgtgccgcttcctctcgtgttgggctcaggtggtttctcacagtccatctgtaaggtaacagcacactaataggagcttcagtgggcttatttatgagataacagcctactcattaatacacctacatgtgaaaggacgatcgtcgaaactggtgttttatagtagtagaaatgaactttgaggcatagcttctttgcaattccattgttagcttgtttttttgttttgaagatacatgtcgttagcttacttgttggattgcatgtaaacatgcatcatggttgctttagaaaattttgttgacgttgatgagtacatttctctatcgcagtacaagatatgtaatgatatagtacttgcatgctcgtgctttcttgcatatgattgcttttcgaaagggaatataaatgccctcgaaaatttacttatcccttgcaatgatctctggtttaagcatagattatttcaaggacttccaaagaaaatgatttgacattcttttttccccttttttcagctagcagccatgccgaagaagaagcgcaaggtcgggggcgggggctcaggcgggggcgggagcggcggcgggggctctgggggcggcggcagcggcgggggcggcagcgggggcggcgggtcgatgagcaagctggagaagttcacgaactgctactccctcagcaagaccctgaggttcaaggcgatcccggtcggcaagacccaggagaacatcgacaacaagcggctgctggtggaggacgagaagagggctgaggactacaagggcgtgaagaagctcctggaccgctactacctgtccttcatcaacgacgtgctccacagcatcaagctcaagaacctgaacaactacatcagcctcttcaggaagaagacgcgcaccgagaaggagaacaaggagctcgagaacctggagatcaacctgaggaaggagatcgccaaggcgttcaagggcaacgagggctacaagtccctcttcaagaaggacatcatcgagaccatcctcccggagttcctggacgacaaggacgagatcgccctggtcaactccttcaacggcttcaccacggcgttcaccggcttcttcgacaaccgcgagaacatgttcagcgaggaggccaagtccacgagcatcgcgttcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtgcatcaacgagaacctcacccgctacatctccaacatggacatcttcgagaaggtcgacgcgatcttcgacaagcacgaggtgcaggagatcaaggagaagatcctgaacagcgactacgacgtcgaggacttcttcgagggcgagttcttcaacttcgtcctcacgcaggagggcatcgacgtgtacaacgccatcatcggtggcttcgtgaccgagtccggcgagaagatcaagggcctgaacgagtacatcaacctctacaaccagaagaccaagcagaagctgccgaagttcaagcccctgtacaagcaggtgctctccgacagggagtccctcagcttctacggcgagggctacacgagcgacgaggaggtcctggaggtgttccgcaacaccctcaacaagaacagcgagatcttctccagcatcaagaagctcgagaagctgttcaagaacttcgacgagtactccagcgccggcatcttcgtcaagaacggcccggcgatctccacgatcagcaaggacatcttcggcgagtggaacgtgatccgcgacaagtggaacgccgagtacgacgacatccacctcaagaagaaggcggtggtcaccgagaagtacgaggacgacaggcgcaagtccttcaagaagatcggctccttcagcctcgagcagctgcaggagtacgccgacgcggacctgagcgtggtcgagaagctcaaggagatcatcatccagaaggtcgacgagatctacaaggtgtacggctccagcgagaagctcttcgacgcggacttcgtcctcgagaagtccctgaagaagaacgacgccgtggtcgcgatcatgaaggacctcctggactccgtgaagagcttcgagaattacatcaaggccttcttcggcgagggcaaggagaccaacagggacgagtccttctacggcgacttcgtcctggcctacgacatcctcctgaaggtggaccacatctacgacgcgatccgcaactacgtgacccagaagccgtacagcaaggacaagttcaagctctacttccagaacccccagttcatgggcggctgggacaaggacaaggagaccgactacagggcgaccatcctgcgctacggcagcaagtactacctcgccatcatggacaagaagtacgcgaagtgcctgcagaagatcgacaaggacgacgtcaacggcaactacgagaagatcaactacaagctcctgccgggccccaacaagatgctcccgaaggtgttcttctccaagaagtggatggcctactacaaccccagcgaggacatccagaagatctacaagaacggcacgttcaagaagggcgacatgttcaacctgaacgactgccacaagctcatcgacttcttcaaggactccatcagccgctacccgaagtggtccaacgcctacgacttcaacttcagcgagaccgagaagtacaaggacatcgcgggcttctaccgcgaggtcgaggagcagggctacaaggtgtccttcgagtccgccagcaagaaggaggtcgacaagctggtggaggagggcaagctctacatgttccagatctacaacaaggacttctccgacaagagccacggcacgcccaacctgcacaccatgtacttcaagctcctgttcgacgagaacaaccacggccagatcaggctgtccggcggcgccgagctcttcatgaggagggcgagcctgaagaaggaggagctggtggtccaccccgctaacagcccaatcgcgaacaagaacccggacaaccccaagaagaccacgaccctgtcctacgacgtgtacaaggacaagaggttcagcgaggaccagtacgagctccacatcccgatcgcgatcaacaagtgccccaagaacatcttcaagatcaacaccgaggtccgcgtgctcctgaagcacgacgacaacccctacgtgatcggcatcgacaggggcgagaggaacctcctgtacatcgtggtcgtggacggcaagggcaacatcgtggagcagtactccctcaacgagatcatcaacaacttcaacggcatcaggatcaagacggactaccacagcctcctggacaagaaggagaaggagaggttcgaggcccgccagaactggacctccatcgagaacatcaaggagctgaaggcgggctacatcagccaggtcgtgcacaagatctgcgagctcgtcgagaagtacgacgccgtgatcgccctcgaggacctgaactccggcttcaagaacagccgcgtcaaggtggagaagcaggtctaccagaagttcgagaagatgctcatcgacaagctgaactacatggtggacaagaagtccaacccctgcgctacgggcggcgcgctgaagggctaccagatcaccaacaagttcgagagcttcaagtccatgagcactcagaacggcttcatcttctacatcccggcgtggctcacgtccaagatcgaccccagcaccggcttcgtcaacctcctgaagacgaagtacacctccatcgccgacagcaagaagttcatctccagcttcgaccgcatcatgtatgtgccggaggaggacctgttcgagttcgccctcgactacaagaacttctcccgcacggacgcggactacatcaagaagtggaagctgtacagctacggcaaccgcatccgcatcttcaggaaccccaagaagaacaacgtcttcgactgggaggaggtgtgcctgacctccgcgtacaaggagctcttcaacaagtacggcatcaactaccagcagggcgacatcagggctctcctgtgcgagcagagcgacaaggccttctactccagcttcatggcgctgatgtccctcatgctgcagatgaggaactcgatcaccggcaggacggacgtggacttcctcatctccccggtgaagaacagcgacggcatcttctacgactccaggaactacgaggcccaggagaacgcgatcctcccaaagaacgcggacgccaacggcgcctacaacatcgccaggaaggtcctctgggctatcggccagttcaagaaggcggaggacgagaagctggacaaggtgaagatcgccatcagcaacaaggagtggctcgagtacgcccagacctcggtcaagcacggcagcccgaagaagaagcgcaaggtgtccggcggcagctccggcggcagcccgaagaagaagcgcaaagtgtgattaattaaggagagggtcgatggatcatgacgtacgcgtaccatatggttgctgtgcctgcccccatcgatcgcgagcaatggcacgctcatgcaagtgatcattgctccctgttggttaaaccctagcctatgttcatggcgtcagcaactaagctaaactattgttatgtttgcaagaaagggtaaacccgctagctgtgtaatcttgtccagctaccatgtatgcttgttactgcccagttacccttgaaatctaggggcgcttttggtgagagggtgcagtttactttaaacatggttcgtgacttgctgtaaatagtagtattaatcgatttgggcatctagtaagtaaattaagggtgtgtttgatttggcttttactctctaaaagttaaaagctaaattaaagagctggatctagaaaatagacttttctaaaagccgactttgtgatggtgcaaaattgaaagcgtcttagcttttggctaaagagttgtttggtttgactcttttgtcctctaaaaaccaaaagtcaaaccaaagagctagatctaaaaacacgtttttttttctttctaaaaatcgtctttctcatttggacctacttttagtgtcttttaaagcatctttggacctactttttacggcttttaatggtttcactaaatggtttttaactttttaacaactcacaatctacatcaatttttttttcatagcttgtagtctacaacaacattttttacaacctacaatctaaccaaatagaccctaactaatgggagaatgaaaaaaaaatcgctcgatcttatgctattgatgcatgacatgtgaaaccatgaggctcatttttgaacgattggatacataaacctagcgatgcttcactgtgacatccggcgcatatctgtatgtttagtgtactttgtttctgtagtttttccttaagtgtagtgcaacttcttatctggtgttttattcggtgtatcgttgtaatcatctagtattcgaagaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggggatccaaattactgatgagtccgtgaggacgaaacgagtaagctcgtctaatttctactaagtgtagatatttgcgaaatgcgcagggccctggccggcatggtcccagcctcctcgctggcgccggctgggcaacatgcttcggcatggcgaatgggactagtttaaattactgatgagtccgtgaggacgaaacgagtaagctcgtctaatttctactaagtgtagatcttggtctgcaggaagggcattgggccggcatggtcccagcctcctcgctggcgccggctgggcaacatgcttcggcatggcgaatgggactagactaaattactgatgagtccgtgaggacgaaacgagtaagctcgtctaatttctactaagtgtagatcccactccatgttgattacctgtggccggcatggtcccagcctcctcgctggcgccggctgggcaacatgcttcggcatggcgaatgggactaacgtaaattactgatgagtccgtgaggacgaaacgagtaagctcgtctaatttctactaagtgtagatatccattggaacacacgcaactgggccggcatggtcccagcctcctcgctggcgccggctgggcaacatgcttcggcatggcgaatgggactaagctaaattactgatgagtccgtgaggacgaaacgagtaagctcgtctaatttctactaagtgtagatagcaggatcctccggacaatttcggccggcatggtcccagcctcctcgctggcgccggctgggcaacatgcttcggcatggcgaatgggactagattaaattactgatgagtccgtgaggacgaaacgagtaagctcgtctaatttctactaagtgtagattccaatcaacattcaatgcccttggccggcatggtcccagcctcctcgctggcgccggctgggcaacatgcttcggcatggcgaatgggactagcgtaaattactgatgagtccgtgaggacgaaacgagtaagctcgtctaatttctactaagtgtagattgatcccgtgaagcctatttttaggccggcatggtcccagcctcctcgctggcgccggctgggcaacatgcttcggcatggcgaatgggactaactagaaattactgatgagtccgtgaggacgaaacgagtaagctcgtctaatttctactaagtgtagatgggaaagaccgaggagaagatctggccggcatggtcccagcctcctcgctggcgccggctgggcaacatgcttcggcatggcgaatgggacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccggaccgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagctaaaccatgcagaagctgatcaacagcgtgcagaactacgcctggggcagcaagaccgccctgaccgagctgtacggcatggagaaccccagcagccagccaatggccgagctgtggatgggcgcccaccccaaaagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatcccaatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagactccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagactactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgataggattacctagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctgctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgatgagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 5 - DNA, 18160 bp, synthetic construct
  source 1..18160: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcccctaaatagaccatgccgaagaagaagcgcaaggtcgggggcgggggctcaggcgggggcgggagcggcggcgggggctctgggggcggcggcagcggcgggggcggcagcgggggcggcgggtcgatgagcaagctggagaagttcacgaactgctactccctcagcaagaccctgaggttcaaggcgatcccggtcggcaagacccaggagaacatcgacaacaagcggctgctggtggaggacgagaagagggctgaggactacaagggcgtgaagaagctcctggaccgctactacctgtccttcatcaacgacgtgctccacagcatcaagctcaagaacctgaacaactacatcagcctcttcaggaagaagacgcgcaccgagaaggagaacaaggagctcgagaacctggagatcaacctgaggaaggagatcgccaaggcgttcaagggcaacgagggctacaagtccctcttcaagaaggacatcatcgagacgatcctcccggagttcctggacgacaaggacgagatcgccctggtcaactccttcaacggcttcaccacggcgttcaccggcttcttcgacaaccgcgagaacatgttcagcgaggaggccaagtccacgagcatcgcgttcaggtgcatcaacgagaacctcacccgctacatctccaacatggacatcttcgagaaggtcgacgcgatcttcgacaagcacgaggtgcaggagatcaaggagaagatcctgaacagcgactacgacgtcgaggacttcttcgagggcgagttcttcaacttcgtcctcacgcaggagggcatcgacgtgtacaacgccatcatcggtggcttcgtgaccgagtccggcgagaagatcaagggcctgaacgagtacatcaacctctacaaccagaagaccaagcagaagctgccgaagttcaagcccctgtacaagcaggtgctctccgacagggagtccctcagcttctacggcgagggctacacgagcgacgaggaggtcctggaggtgttccgcaacaccctcaacaagaacagcgagatcttctccagcatcaagaagctcgagaagctgttcaagaacttcgacgagtactccagcgccggcatcttcgtcaagaacggcccggcgatctccacgatcagcaaggacatcttcggcgagtggaacgtgatccgcgacaagtggaacgccgagtacgacgacatccacctcaagaagaaggcggtggtcaccgagaagtacgaggacgacaggcgcaagtccttcaagaagatcggctccttcagcctcgagcagctgcaggagtacgccgacgcggacctgagcgtggtcgagaagctcaaggagatcatcatccagaaggtcgacgagatctacaaggtgtacggctccagcgagaagctcttcgacgcggacttcgtcctcgagaagtccctgaagaagaacgacgccgtggtcgcgatcatgaaggacctcctggactccgtgaagagcttcgagaattacatcaaggccttcttcggcgagggcaaggagacgaacagggacgagtccttctacggcgacttcgtcctggcctacgacatcctcctgaaggtggaccacatctacgacgcgatccgcaactacgtgacccagaagccgtacagcaaggacaagttcaagctctacttccagaacccccagttcatgggcggctgggacaaggacaaggagacggactacagggcgaccatcctgcgctacggcagcaagtactacctcgccatcatggacaagaagtacgcgaagtgcctgcagaagatcgacaaggacgacgtcaacggcaactacgagaagatcaactacaagctcctgccgggccccaacaagatgctcccgaaggtgttcttctccaagaagtggatggcctactacaaccccagcgaggacatccagaagatctacaagaacggcacgttcaagaagggcgacatgttcaacctgaacgactgccacaagctcatcgacttcttcaaggactccatcagccgctacccgaagtggtccaacgcctacgacttcaacttcagcgagaccgagaagtacaaggacatcgcgggcttctaccgcgaggtcgaggagcagggctacaaggtgtccttcgagtccgccagcaagaaggaggtcgacaagctggtggaggagggcaagctctacatgttccagatctacaacaaggacttctccgacaagagccacggcacgcccaacctgcacaccatgtacttcaagctcctgttcgacgagaacaaccacggccagatcaggctgtccggcggcgccgagctcttcatgaggagggcgagcctgaagaaggaggagctggtggtccaccccgctaacagcccaatcgcgaacaagaacccggacaaccccaagaagaccacgaccctgtcctacgacgtgtacaaggacaagaggttcagcgaggaccagtacgagctccacatcccgatcgcgatcaacaagtgccccaagaacatcttcaagatcaacaccgaggtccgcgtgctcctgaagcacgacgacaacccctacgtgatcggcatcgacaggggcgagaggaacctcctgtacatcgtggtcgtggacggcaagggcaacatcgtggagcagtactccctcaacgagatcatcaacaacttcaacggcatcaggatcaagacggactaccacagcctcctggacaagaaggagaaggagaggttcgaggcccgccagaactggacctccatcgagaacatcaaggagctgaaggcgggctacatcagccaggtcgtgcacaagatctgcgagctcgtcgagaagtacgacgccgtgatcgccctcgaggacctgaactccggcttcaagaacagccgcgtcaaggtggagaagcaggtctaccagaagttcgagaagatgctcatcgacaagctgaactacatggtggacaagaagtccaacccctgcgctacgggcggcgcgctgaagggctaccagatcaccaacaagttcgagagcttcaagtccatgagcactcagaacggcttcatcttctacatcccggcgtggctcacgtccaagatcgaccccagcaccggcttcgtcaacctcctgaagacgaagtacacctccatcgccgacagcaagaagttcatctccagcttcgaccgcatcatgtatgtgccggaggaggacctgttcgagttcgccctcgactacaagaacttctcccgcacggacgcggactacatcaagaagtggaagctgtacagctacggcaaccgcatccgcatcttcaggaaccccaagaagaacaacgtcttcgactgggaggaggtgtgcctgacctccgcgtacaaggagctcttcaacaagtacggcatcaactaccagcagggcgacatcagggctctcctgtgcgagcagagcgacaaggccttctactccagcttcatggcgctgatgtccctcatgctgcagatgaggaactcgatcaccggcaggacggacgtggacttcctcatctccccggtgaagaacagcgacggcatcttctacgactccaggaactacgaggcccaggagaacgcgatcctcccaaagaacgcggacgccaacggcgcctacaacatcgccaggaaggtcctctgggctatcggccagttcaagaaggcggaggacgagaagctggacaaggtgaagatcgccatcagcaacaaggagtggctcgagtacgcccagacctcggtcaagcacggcagcccgaagaagaagcgcaaggtgtccggcggcagctccggcggcagcccgaagaagaagcgcaaagtgtgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaacgcgttaactagctaggaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggtgcgcaggtacctaaattactgatgagtccgtgaggacgaaacgagtaagctcgtctaatttctactaagtgtagatatccattggaacacacgcaactgggccggcatggtcccagcctcctcgctggcgccggctgggcaacatgcttcggcatggcgaatgggacgaagaaattactgatgagtccgtgaggacgaaacgagtaagctcgtctaatttctactaagtgtagattgatcccgtgaagcctatttttaggccggcatggtcccagcctcctcgctggcgccggctgggcaacatgcttcggcatggcgaatgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccggaccgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagctaaaccatgcagaagctgatcaacagcgtgcagaactacgcctggggcagcaagaccgccctgaccgagctgtacggcatggagaaccccagcagccagccaatggccgagctgtggatgggcgcccaccccaaaagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatcccaatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagactccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagactactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgataggattacctagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctgctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgatgagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 6 - DNA, 15613 bp, synthetic construct
  source 1..15613: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccctaattagctaaggtaccttatagccgtttctttgttttttttcttctgttgagctccaactttatgaaacgtcgtaagctggtaattatgaaatgtaaggatttggagagagaaaaaaaacgggagggaaaaccatgcatgctgctgacgcgacggccggacgcagacgcaacaatgcccccggtgcggcgttgtcgagcagccactgcaccaccccacgcatcacctgcagtaatctagcgacgggtttttcttatttatttatttatttatttatttttcttctctccctccctccctcagatttgttttcgttttcattaatcgttattaccagcaattaattaactttatctattgatttaccaaaccgcaataaagaatatatatattcttttattaaggtccagtaataagcagcacagaagcgcaggtgcagcagcagcagcgtcagcgcccgaggcgcgcacgagagaaacagaggctgacgaggtggggcccgtgcgggccttgaccaatcggagttcgacaacagcctggccacccacaaacacacactccttcgcctcgcgccggccgtcgtcgcctccctccaccgaacgatccctcctcctcctcctcctcctcctcctcgcatcccaccccaccccaccttctccttaaagctacctgcctacccggcggttgccgccgccgcaatcgatcgaccggaagagaaagagcagctagctagctagcagatcggagcacggcaacaaggcgaaggggcgcggcaaggtacagctgaagcggatagagaacaagataaaccggcaggtgaccttctccaagcgccggaacggcctgctcaagaaggcgcacgagatctccgtcctctgcgttgccgaggtcgccgtcatcgtcttctcccccaagggcaagctctacgagtacgccaccgactcccggtacgtacgaccaccttcaattcgtcacacaggcagtgtgtttgttgaattgaatacgtgcctgctatgctagctgcctgtcagtatggtccatccgttcgccatcagttcatctttctgcttcgtgctgctgttgtgtgtgtgcgccataccatgccatgccatgccatgccaccaacctggctgtgctgccgtttttggctcgcagccgccgatccccgacccgaaagctcatgctgctcttgccgttttcggccaccacccaccatggcctgctgtctggtcctccacgcctgcttccccagttccacttccatgcatggtggtcagttggctggcatggcagcagcagccgccttctggttcgtttgtgctgcctcaggatttgctttctactagctagtgtgctacagccgatcatttcatgctgctgcccgcgtcgatctgctccgctagctgcttcctcgtttcgggaaacctcgcccgccccgccattaactgattcacctgctttccttctgttgcggtgttgcctacgacgactgaaacctttcctttttggggattggcgtctgccgactccactaggcctccttttaattccttgttttatatacatgtacataagcttttgctgatgcagtggtacggtgcctttatttttatcggattacagtacgtgttagattgataacagcacgcaatcagatctagctttgcttgcttccttccttcctcgtcaaatctttctggcccttctcgatttgcgctccatcgtataattaattaaccatcactaatcacactttccccatttttctaaacaaaaaaaagtatcacatgttcctaatcacgcgtgcatgtaatttgctgtttctttctctctctctctcaattaatttgctgcctgccgagaaacacgagctgcatgctggctcaatcagttcctaactgcgaaagtgcagataatgaccccctcggctcggattaatcagtacacatgatcgaggacgcatagatctggcttttccttatctggttcttcgtcttcaattctccgtttcttccctcgcatccttttccgtgttgtttcagatttacaaataaaccatcgtctctctcactttcatctgttgttcttccatgacagtgaaaaatactgttttttcctcgggtgcatggaaaaattaaaattaaaggcccgtgtttttttctctttggatgtgttcttataggatggatacactatgtgcatccgggttttgtggtgatgtaacatagtaaagcagctccgaggtactgggttctatttcatattccccccccccccaaatcaaaaaagttatctttgctcgaaggtctactcgtttttttttctggcttttatacgaattctatgacatgctagtatgggtaaaaacgaaatggtgattctcattcgtatatgcactattatattctttgcatgcacttttattacaaaagctgcatggacgtgttacacattcacggagataacaattgagcagtagattagtttctgtcttcttaattagctaggcaggctcagaggtgaaactgcagctattaggttgagtaacacagctagtgctgtattcatgaagcttcggtttgactcagttttgcagtagcactggttgcaactcgctaaaccatttaacttccatgtacatgatgtctttgtaggagtttgtgttcattcttcttattttgacatttcaagtcattcagtacgtaactactaaaataagtgtcttgcagagtcagtctcaagataacttgttggtcttggcttattagctaagatgcaatgcatcattacttgccttactgtgatggctaaaccaatatcacatgcactgtgtgctttttcaagcattccacatccatacatacctagtatccctgactttttaatgacccctgatgatagaacatgcattgtttgtatattacctgttagtgttcttaacataaaattttacaaaagaagctctattacgaggatgttatgctgcttttgcaaaggaaactagctaggcagactggtgctttttaaataaacaattaaaatcttgtgaacccaaacactacttacagatcgaaaattacagtaagacgaaagtaaatggcaactagtacaatactcgtacatgcgttgcgacggcacataaaaacaaatattacgttttatcttagccaaagcgtctagaaaggtatgagttgaaaagaagactttttatagcttactcggtcgctgttcttcctttagctagcaagttaatactatatgggagcgctacgcagtgtgccgcttcctctcgtgttgggctcaggtggtttctcacagtccatctgtaaggtaacagcacactaataggagcttcagtgggcttatttatgagataacagcctactcattaatacacctacatgtgaaaggacgatcgtcgaaactggtgttttatagtagtagaaatgaactttgaggcatagcttctttgcaattccattgttagcttgtttttttgttttgaagatacatgtcgttagcttacttgttggattgcatgtaaacatgcatcatggttgctttagaaaattttgttgacgttgatgagtacatttctctatcgcagtacaagatatgtaatgatatagtacttgcatgctcgtgctttcttgcatatgattgcttttcgaaagggaatataaatgccctcgaaaatttacttatcccttgcaatgatctctggtttaagcatagattatttcaaggacttccaaagaaaatgatttgacattcttttttccccttttttcagcggatccgcgtaccccgccgcgccgcacacgagcgcggccttcctcgctagcttcgcgacgccgctccccgcgcgccgcgcctcgcccccgacccggcgcagcgcggtgtcggtcgccgccatcgccaccaccaccacctgcgtcttcgccgacgccccccgtgggatcaaggaggagcaggcggactcgggcgctcaggcggcggcggctgcggctgcgcggagggacgtcagcccggacaccgtggcctccatcatcctcggcggtggcgccggcacgcgccacttcccgctcacgcgtacaagggccaagcccgcggtgcccgtcgggggatgctacaggctcatcgacatcccgatgagcaactgtaactagtcgacgtcagtcgcgctcgcgcattgccgtgaggtccgcgtgcttccgacttccaacaattggacatgctagccgccgccaagggcctaggccacgctggtgtcaagcgtggcgtgatctgtgttgcgttcgcgtgccgtgccgttccgtggtagggcgggtcatgggtcaatcacattttcgggttgtcacctgtggcagtcgactagtccagttgctcatcgggatgtcgatgagcctgtagcatcccccgacgggcaccgcgggcttggcccttgtacgcgtgagcgggaagtggcgcgtgccggcgccaccgccgaggatgatggaggccacggtgtccgggctgacgtccctccgcgcagccgcagccgccgccgcctgagcgcccgagtccgcctgctcctccttgatcccacggggggcgtcggcgaagacgcaggtggtggtggtggcgatggcggcgaccgacaccgcgctgcgccgggtcgggggcgaggcgcggcgcgcggggagcggcgtcgcgaagctagcgaggaaggccgcgctcgtgtgcggcgcggcggggtacgccctaggagctcaagctacggagagggtcgatggatcatgacgtacgcgtaccatatggttgctgtgcctgcccccatcgatcgcgagcaatggcacgctcatgcaagtgatcattgctccctgttggttaaaccctagcctatgttcatggcgtcagcaactaagctaaactattgttatgtttgcaagaaagggtaaacccgctagctgtgtaatcttgtccagctaccatgtatgcttgttactgcccagttacccttgaaatctaggggcgcttttggtgagagggtgcagtttactttaaacatggttcgtgacttgctgtaaatagtagtattaatcgatttgggcatctagtaagtaaattaagggtgtgtttgatttggcttttactctctaaaagttaaaagctaaattaaagagctggatctagaaaatagacttttctaaaagccgactttgtgatggtgcaaaattgaaagcgtcttagcttttggctaaagagttgtttggtttgactcttttgtcctctaaaaaccaaaagtcaaaccaaagagctagatctaaaaacacgtttttttttctttctaaaaatcgtctttctcatttggacctacttttagtgtcttttaaagcatctttggacctactttttacggcttttaatggtttcactaaatggtttttaactttttaacaactcacaatctacatcaatttttttttcatagcttgtagtctacaacaacattttttacaacctacaatctaaccaaatagaccctaactaatgggagaatgaaaaaaaaatcgctcgatcttatgctattgatgcatgacatgtgaaaccatgaggctcatttttgaacgattggatacataaacctagcgatgcttcactgtgacatccggcgcatatctgtatgtttagtgtactttgtttctgtagtttttccttaagtgtagtgcaacttcttatctggtgttttattcggtgtatcgttgtaatcatctagtacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatctccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 7 - DNA, 18745 bp, synthetic construct
  source 1..18745: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaacaaagcttggtaccattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggatcgcggccgctcatatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggacccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaataagcgatcgctttgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaactagtggtgcctgatgagtccgtgaggacgaaacgagtaagctcgtcgcaccacagcatagcaagttaaaataaggctagtccgttatcaacttgaaaaagtggcaccgagtcggtgcttggccggcatggtcccagcctcctcgctggcgccggctgggcaacatgcttcggcatggcgaatgggactaattaagtcgacagtcgtctgatgagtccgtgaggacgaaacgagtaagctcgtcacgactactacgcgctgggggttttagagctatgctgtggtgggccggcatggtcccagcctcctcgctggcgccggctgggcaacatgcttcggcatggcgaatgggactagttgactgagggcctgatgagtccgtgaggacgaaacgagtaagctcgtcgccctcgtacttggtccagtgttttagagctatgctgtggtgggccggcatggtcccagcctcctcgctggcgccggctgggcaacatgcttcggcatggcgaatgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcaatagactacggaccgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagctaaaccatgcagaagctgatcaacagcgtgcagaactacgcctggggcagcaagaccgccctgaccgagctgtacggcatggagaaccccagcagccagccaatggccgagctgtggatgggcgcccaccccaaaagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatcccaatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagactccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagactactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgataggattacctagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctgctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgatgagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 8 - DNA, 15575 bp, synthetic construct
  source 1..15575: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggaccctaattagctaaggtaccttatagccgtttctttgttttttttcttctgttgagctccaactttatgaaacgtcgtaagctggtaattatgaaatgtaaggatttggagagagaaaaaaaacgggagggaaaaccatgcatgctgctgacgcgacggccggacgcagacgcaacaatgcccccggtgcggcgttgtcgagcagccactgcaccaccccacgcatcacctgcagtaatctagcgacgggtttttcttatttatttatttatttatttatttttcttctctccctccctccctcagatttgttttcgttttcattaatcgttattaccagcaattaattaactttatctattgatttaccaaaccgcaataaagaatatatatattcttttattaaggtccagtaataagcagcacagaagcgcaggtgcagcagcagcagcgtcagcgcccgaggcgcgcacgagagaaacagaggctgacgaggtggggcccgtgcgggccttgaccaatcggagttcgacaacagcctggccacccacaaacacacactccttcgcctcgcgccggccgtcgtcgcctccctccaccgaacgatccctcctcctcctcctcctcctcctcctcgcatcccaccccaccccaccttctccttaaagctacctgcctacccggcggttgccgccgccgcaatcgatcgaccggaagagaaagagcagctagctagctagcagatcggagcacggcaacaaggcgaaggggcgcggcaaggtacagctgaagcggatagagaacaagataaaccggcaggtgaccttctccaagcgccggaacggcctgctcaagaaggcgcacgagatctccgtcctctgcgttgccgaggtcgccgtcatcgtcttctcccccaagggcaagctctacgagtacgccaccgactcccggtacgtacgaccaccttcaattcgtcacacaggcagtgtgtttgttgaattgaatacgtgcctgctatgctagctgcctgtcagtatggtccatccgttcgccatcagttcatctttctgcttcgtgctgctgttgtgtgtgtgcgccataccatgccatgccatgccatgccaccaacctggctgtgctgccgtttttggctcgcagccgccgatccccgacccgaaagctcatgctgctcttgccgttttcggccaccacccaccatggcctgctgtctggtcctccacgcctgcttccccagttccacttccatgcatggtggtcagttggctggcatggcagcagcagccgccttctggttcgtttgtgctgcctcaggatttgctttctactagctagtgtgctacagccgatcatttcatgctgctgcccgcgtcgatctgctccgctagctgcttcctcgtttcgggaaacctcgcccgccccgccattaactgattcacctgctttccttctgttgcggtgttgcctacgacgactgaaacctttcctttttggggattggcgtctgccgactccactaggcctccttttaattccttgttttatatacatgtacataagcttttgctgatgcagtggtacggtgcctttatttttatcggattacagtacgtgttagattgataacagcacgcaatcagatctagctttgcttgcttccttccttcctcgtcaaatctttctggcccttctcgatttgcgctccatcgtataattaattaaccatcactaatcacactttccccatttttctaaacaaaaaaaagtatcacatgttcctaatcacgcgtgcatgtaatttgctgtttctttctctctctctctcaattaatttgctgcctgccgagaaacacgagctgcatgctggctcaatcagttcctaactgcgaaagtgcagataatgaccccctcggctcggattaatcagtacacatgatcgaggacgcatagatctggcttttccttatctggttcttcgtcttcaattctccgtttcttccctcgcatccttttccgtgttgtttcagatttacaaataaaccatcgtctctctcactttcatctgttgttcttccatgacagtgaaaaatactgttttttcctcgggtgcatggaaaaattaaaattaaaggcccgtgtttttttctctttggatgtgttcttataggatggatacactatgtgcatccgggttttgtggtgatgtaacatagtaaagcagctccgaggtactgggttctatttcatattccccccccccccaaatcaaaaaagttatctttgctcgaaggtctactcgtttttttttctggcttttatacgaattctatgacatgctagtatgggtaaaaacgaaatggtgattctcattcgtatatgcactattatattctttgcatgcacttttattacaaaagctgcatggacgtgttacacattcacggagataacaattgagcagtagattagtttctgtcttcttaattagctaggcaggctcagaggtgaaactgcagctattaggttgagtaacacagctagtgctgtattcatgaagcttcggtttgactcagttttgcagtagcactggttgcaactcgctaaaccatttaacttccatgtacatgatgtctttgtaggagtttgtgttcattcttcttattttgacatttcaagtcattcagtacgtaactactaaaataagtgtcttgcagagtcagtctcaagataacttgttggtcttggcttattagctaagatgcaatgcatcattacttgccttactgtgatggctaaaccaatatcacatgcactgtgtgctttttcaagcattccacatccatacatacctagtatccctgactttttaatgacccctgatgatagaacatgcattgtttgtatattacctgttagtgttcttaacataaaattttacaaaagaagctctattacgaggatgttatgctgcttttgcaaaggaaactagctaggcagactggtgctttttaaataaacaattaaaatcttgtgaacccaaacactacttacagatcgaaaattacagtaagacgaaagtaaatggcaactagtacaatactcgtacatgcgttgcgacggcacataaaaacaaatattacgttttatcttagccaaagcgtctagaaaggtatgagttgaaaagaagactttttatagcttactcggtcgctgttcttcctttagctagcaagttaatactatatgggagcgctacgcagtgtgccgcttcctctcgtgttgggctcaggtggtttctcacagtccatctgtaaggtaacagcacactaataggagcttcagtgggcttatttatgagataacagcctactcattaatacacctacatgtgaaaggacgatcgtcgaaactggtgttttatagtagtagaaatgaactttgaggcatagcttctttgcaattccattgttagcttgtttttttgttttgaagatacatgtcgttagcttacttgttggattgcatgtaaacatgcatcatggttgctttagaaaattttgttgacgttgatgagtacatttctctatcgcagtacaagatatgtaatgatatagtacttgcatgctcgtgctttcttgcatatgattgcttttcgaaagggaatataaatgccctcgaaaatttacttatcccttgcaatgatctctggtttaagcatagattatttcaaggacttccaaagaaaatgatttgacattcttttttccccttttttcagcggatcctcgcgcgtctaccccaccgaggccatctacgccaacgccggcgacttcctcttcaacaacgccaccgccgcgcgggtcacggccacgaagctcgtcgtccacgagatggactcgtcatacaaccacgactacttggcgccggtggcagacatctgatcgatgcaatgcaaccacgccactctgcaccgcaacaaattttccaacacacataactataccaagcaattcccatctgtcaaattttcttattaactagtcgacgtacgttacgatcctctctgtctctctctctctctctctgctgctgctgcgctgctgcatttggtttgggttgcatcccaactcaacatccccttccaaacgactatggattagatcgatttgaaccatatgaacgaattaattgtccacgcatttgattcctttcacgtgcatttcatccaacgagtttcccttttttttttctttctcatccacatacaaattccagatacccccgggccctccgggattccacccaaccaccaggcctaccgttgtgtattcttcttttgtttttctgcccttctcgtgcctgacttgtttggtccaagtcgtcgtcgcttcgtaataataataataaattgtaaattatacgcttgcagtcgactagtcaataagaaaatttgacagatgggaattgcttggtatagttatgtgtgttggaaaatttgttgcggtgcagagtggcgtggttgcattgcatcgatcagatgtctgccaccggcgccaagtagtcgtggttgtatgacgagtccatctcgtggacgacgagcttcgtggccgtgacccgcgcggcggtggcgttgttgaagaggaagtcgccggcgttggcgtagatggcctcggtggggtagacgcgcgacctaggagctcaagctacggagagggtcgatggatcatgacgtacgcgtaccatatggttgctgtgcctgcccccatcgatcgcgagcaatggcacgctcatgcaagtgatcattgctccctgttggttaaaccctagcctatgttcatggcgtcagcaactaagctaaactattgttatgtttgcaagaaagggtaaacccgctagctgtgtaatcttgtccagctaccatgtatgcttgttactgcccagttacccttgaaatctaggggcgcttttggtgagagggtgcagtttactttaaacatggttcgtgacttgctgtaaatagtagtattaatcgatttgggcatctagtaagtaaattaagggtgtgtttgatttggcttttactctctaaaagttaaaagctaaattaaagagctggatctagaaaatagacttttctaaaagccgactttgtgatggtgcaaaattgaaagcgtcttagcttttggctaaagagttgtttggtttgactcttttgtcctctaaaaaccaaaagtcaaaccaaagagctagatctaaaaacacgtttttttttctttctaaaaatcgtctttctcatttggacctacttttagtgtcttttaaagcatctttggacctactttttacggcttttaatggtttcactaaatggtttttaactttttaacaactcacaatctacatcaatttttttttcatagcttgtagtctacaacaacattttttacaacctacaatctaaccaaatagaccctaactaatgggagaatgaaaaaaaaatcgctcgatcttatgctattgatgcatgacatgtgaaaccatgaggctcatttttgaacgattggatacataaacctagcgatgcttcactgtgacatccggcgcatatctgtatgtttagtgtactttgtttctgtagtttttccttaagtgtagtgcaacttcttatctggtgttttattcggtgtatcgttgtaatcatctagtacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatctccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 9 - DNA, 6737 bp, Zea mays
  source 1..6737: mol_type = genomic DNA
gaaaaggtgacggaaatagtgaggagcggagaacaccggaatgatccatcctcttgtgctttccctgcccttccccgctataatatcgcgccctcgtcgccatcgtcaccacaccaccactccctcactgccctctcactcccgatccctgcaccactaccgcctcctccgcttcacccctctcgtcgcctcttgcggcgaccggcggcggatcgtccgcggcgcaagcgcaaccatggggctcttcaccgtgacgaagaaggccaccacccccttcgaaggccagaagcccggtacctccggcctccgcaagaaggttcccttcccgacttttccttatctatcccccgatctccgggccccagtccggatctctctgtttctgcgacgcggcgggttcgtgctcgattctggtgtctcgaatccgccgcgcgacagcctcgcttcccgccacctcgcggatctcgccgggcggcttttcgtgcaggatctcggggctctgttctatccgtccttctgcttggatctactgggaaaccgttgtcggatgggtgttgttcgggggcatgccttgcctgctggcttcatccgtttggttcctataggacattggacctgcgcggggctggaacatgcttgcatggctttgatccatacgatttgcggggctggaatatacttgcatggctttgatccatacgatttgtactcagtactcgcattaatcagattttgctaattgttaccggatctcgctgattcaattactggtttagaagataacgatgccctctgttttactagctcctgcagaatagtgggtatggttttttggtctctcaatggtaactcctccattcgagataatgttcacatgaaaattgttctagtagcattgatgcatatggttgggttggttcgacattgattaaattcaggctcatcgtatgttcagattcatggaattcatttcccatcctggttcaggcttgagtggatcgtgtttccaaaatgggatgaggggcagagtattttgattttgtagttgctacccttggcttataaatgtagttcgacttcgattcataaaggtggaaaaattatggtgcaagttatttaaatttgatatcgacctttttgttgattgcatgtgtagcaccactctgtaaattctgtgcacgtctcaaattgcttgaggtgtgatccgagtattttgtactagtgtgtatggatctccagcggcagcagtcaaacaatacccattctgaatttgatcaatctaatgttttcgcacaatattgattgcctttacatggttgcatctttgttgtttaattgttatacattattaagtaggaaaagatgaaaataatacaatttattattagtggtatagataaaaaaattattcctccagtcaaggcagccatgtccattatatctgtctgcacagcttgaactgtgtaactcctagtgtttatgcacatgcattattttctttgatgctaggcatatcatttgttagctagttagccttcctttgatgggaattaataaaatgtatgttaatcaggatgagtttacttctacatcaaggaaaaaagcatgaatgatcactgatttcagatgcaaacgattttgtctacagatactttgttactttggatgcttgctttttatgtatagcatagtacaattattattgttttttttcttataattaatattttgttaattaatattgccaggttactgtattccagcagcctcattatctgcagaactttgtccagtcaacattcaatgcccttcctgcagaccaagtaaaaggtaacctacttatcagattatcagtcctgtttcaaacaaggtttccaggataccgcaaggtttagtataaattattattgtgcagttttcatgtatatgtgaaattgtaaatgggaaatgtagttgccatagccaaatttttatgacctccgttctctcttttcagttgttgctaattagtcatgtctcctttcctttcttataggtgcaaccattgttgtctctggtgatggccgctatttctcaaaagatgctgttcaggtgatttagaacagtaccatctcccttatatattttctaaccccatcttataatttttatactgctttgatagtagctattgtagacttgaattccgatgcagatgtagcattgttgtcttctccctgaggtcgagtttttttaatagacttggtgttcatgaactacattgaatcccattgattttgcttgttacagatcataacaaaaatggctgctgccaatggagtaagacgtgtttgggttggacaaaacagtctcatgtctactcctgctgtatctgctgtcatccgtgaaagaattggtgcagatgtaagtttgattcttaatgaaggacagtctttagcagtatgaatatttacttgttattcctttatactgtctaatgaataacctgactgttggtttataaacacatgaacaaaagggatcaaaggctactggtgccttcatcttgacagcgagccataacccaggtggtcctacggaggtaccaagtttactgctagtgtctttgttgcaaatcaagtgccacacatgctctatgttattgaatacgcctcactgaactttccaggactttggtatcaaatacaatatgggaaatggtggacctgcccctgaatccgttaccgacaagattttctctaatacaacgacaatctctgaatacctcatctctgaagaccttccagatgtatgattcagtgccaacctgccaagtattttctctgtttctttgacaggttagtggaatattcattttctctcactttactactgcaggttgatatttctgttgtcggtgtcaccagcttcagtggacccgaaggcccctttgatgtggatgtctttgactctagtgtaaattacataaagttaatgaagtgagtagcttgttctttcctgtttcctttcattgaagtgagaattccatgctttatatcattacttgttacttggatcatgttatgtagcaagggaacctaatgatttaattaccaccatcaatttcaggacaatttttgacttcgaagcaataaaaaagctactgacctccccaaagtttacattctggtattcatctatgactaaaaccatgttaacattaccgtaatagtattattgtaacacatccaaaatcttgctttgcacttacagttatgatgcgctccatggtgttgctggagcttatgccaaacacatctttgtggaagagcttggtgctgatgaaagctcactgttgaattgtgtcccaaaagtaatgagttcttctattgtcacaagtttctacttaaaatatggcttgggaattcatcacttatgtttttgcaggaggactttggaggtggtcatccggatcctaacctcacctatgcaaaagagttggttgaacggatgggtcttggaaagtcatcctcaaatgttgaacctcctgaatttggtgctgcagctgatggagatgctgaccgcaacatgattctgggtaaaaggtatgatctgttcgagtatcttattatgtagaaagtttgttttggattctagataagcacaaggaattcatgatgcttcccctttgccgtgtttatagattctttgtgacaccatcggactctgttgccattatagcggccaatgctgttcaatcaattccttactttgcttctggcctgaagggagttgccaggtaaaaagctctcctccatattgtgctgctaatataacatctagatatatttttactgtaagaactctcactgtgggcaccaagcggtaagcaccactgcagtctgcaataaaaatgcatgtttccctctacattggctctgcatctgcttaagtttggcagcgttctgctcaataaatctgcttaacatatcagcaacaataccatctggaacacagtttctgctaatgccaaccttttgatcccccacttgtatgcagctatctgaccttagatgcatttaaaatttctctatgcactgataggctacttacgacaatctaatgttgtaaatgcaggagcatgccaacatcagctgcccttgatgttgttgcaaagaatttgaatctcaagttctttgaggtaaagtattaagaccttggtttgcaagattttgaatgctagataaatctaacataaaaaacatgtttcaggtgcctactgggtggaaattttttgggaatttgatggatgctggaatgtgctcaatctgtggtgaagaaagctttggcactggtaatatttggcttctggctatcatttctatacatgccaatcacagtagtaaacatgtcaccacctcttgtattgtcatgttagatgggtaaattgtgttatcatgatgtggtatatgaatggggcaaatcacttgtgttctgacagtgagctctctcgcccttttattgagttagtggtcatgaatccaacccatagtaaagtcaaggacttgttggtatttggtatagacattattgagatgtttatatgtactactttggctagggtctgaccacattcgtgagaaagatggcatctgggctgtgcttgcatggctttctattattgctttcaagaataaggacaaccttggaggagataagcttgtcactgttgaagatattgtccgtcagcattgggccacatatggtcgccattattacacacgctatgactatgaggtacttcctttttttcacatgtgccttgcatatatgtaccacatgtttcagatatatatcattctaaatacattagtttgtttgtgaagaatgtcgatgctggggctgctaaggagctgatggcaaacctagtaagcatgcagtcatcactttctgatgttaacaagtaagtggaactttttcaaatgtacaggaatctcaaacttgaactttttagaaccatgtccaagtactcccgtgtatatatcaacttcagaaaattatgatagtactcccgtgtatatatcaattcaagcaatcgcaaacttgaactttttagaaccatgtctaaagtatctctttgtcaggttgatcaaggagatccggtctgatgtttctgaagtagttgcagctgacgagtttgagtacaaggatccagttgatggctctgtgtccaagcaccagggcatccgatacctcttcggagatggttcacgactggtatgtagacctgtattgtttcagatatcctctattttatgtcttgaaccaaaacaaagggtctggtgtgaaaaccttgttccgcagtcagctgtacaatttttttttgcaatattatcagtcagtacatgataagtctgagccttggtcatggatatatttgagatctctgactatgggggtgtttggcttctagggactaatttttagtcccgccattttattttattttattttagtttataaattgccaaatacggaaattaaaactctattttagtttctgtatttgacgggactaagatgttataaaatgaagggactaaaaattagtccctagaaaccaaacaccccctatatcatactggccacaagttgcgctggtgagctaacatggatttttggcgttctaggtgttccgtctatccggaaccggttctgttggtgccaccatccgtgtctacatcgagcaatacgagaaggattcctccaagaccggcagggattcacaggaggcccttgctccactggtaagataatttccccgtccttttgatttacagagctgaatatactaggggtgaagaaacatttggatcatcatccattaccacctctaagaatcagattaggatcgacctctattcctatttatttttgaactaaaattagaattagttaagggtcctaacttagtgtgaagaaacatttggatcgtgatccattaccacccctaaatataccttgggcttatgtgaaactgcatttactaattttgtgtttcatctcaggttgatgttgcgctcaagctctccaagatgcaagagtacactggccgctctgcccccaccgttatcacataaatttttgaagagtgatttacaatgagttgaggcgcttacacaaattttcattcaagcctcttgttctatagttctacttcctgttgcatctcaccaatgaataaaatgtatgtatcagactcgccccgttttttttttgtccatgtactgcagtgcagcggactgagccgctggcacagcatgcggtaataatcttgtcacagtgacagtgctgtaatcgggagcgtgtttcttgtaatcttgttaggaagtgtttctggttttgtttgagcatttgcttatctttttttctgagtactaaaaataatttactcacatgtatgtataccatgtcactaatcttttgatggctgtttagggaacctgggttatgattcgtcgattactgattaaccgttccctgtgtggttacgggctacggcaggggattgtgactgcttgagggttcggttagcttgtgtgctttgtaagggtgagtgtgtgtttgtgtttagagtgtgtgtttgtgttgtgtgtgttctgtttgtcctgggccataggtcctttttctctctactttaatttaatgatatgtagccctcctttgtattcggaaaaaaaaaatccacatggattgag

SEQ ID NO: 10 - DNA, 6008 bp, Zea mays
  source 1..6008: mol_type = genomic DNA
cgaaaaggtgagataaaacgctaggagccgaggagaacaactgaatgatccaacaccgccctcttgtgctctccctgccctcccccgctataataccgccacgccctcgtcgccatcgtcaccacaccaccactccctccctgccctctcactcccgatccctcctccaccaccgcttcctccgcgtcacccatctcgtcgtcgcctcacgaggcgaccagcggcggaccctccgcggcgcaaccatggggctcttcactgtgacgaagaaggccaccacgcccttcgacggccagaagcccggcacctccggcctccgcaagaaggttcccttgcttcccgcctttgccttatctcgccccccggtctccggctaagttcataggggcccagttcggatctctctgtttctgtgacgcggcgggttcgtgctcgtttctggtgcctcggatccgctgcgcgacagccttcgcttcccgccacctcgcggatctcgccgggtttccgcgcaggatctcggggctctgttctatccgtccttctgcttggatctgctgggaaacggttgtcggatgggtgttgttcggagcatgactgctagcttgatccgttggttgcttcaggacgttggacctcacggggtcagaaactcagaatgatctgaactgtatgcttgcatggctttgatccatacgatttgtactcgcattgaccagctttcgttaatcgttgccggacctcgctgattcaattactggcttattgatggtagaagataactgtaactctgttctaccagctcctgcatacttagtggatatggttttttgatctctcaatgattactccattcgagacaatgttcatatgaaaattgttttagcgtcatttagtgcatatatggttgggttggttcgacattcattaaattccagctcatcatatgttgagattcatggaattcatttccatcctggtccaggcttgagtagatcatgtgtttcaaatgggatgaggggcagagtattttgattttgtagttgattctgcacagctcgaacagtgtaactgcctagtgtttatacacatgcattgttttctttgatgctgagcatagcatttgttcctagctagctagcctttccttgatgggaattaagaaagtatatgttaattgggatgaggttattctacatcaagggaaaaaaatctgaatggtcactgattgcagatgaaaatgattttgtcatcagtgacttgttactttaatgtttggtttcatctgtagcattgtacaattcagtataccttttttcttataattaatcttttgttaatattaccaggttactgtattccagcagccccattatctgcagaactttgtccaatcaacattcaatgcccttcctgtggatcaagtaagaggtaacatgcttatcagataaataccattcctgcttcaaacaaggtttttacgataccacaagattttaataaattatcattgtgttgttttcatgtgtatgctgaaattgtaatgggaaatgtagttcccaaaaatttcatgatctcccctgttatttctctgctgttgttccccattgttcacgtcttttcttttttttttttaggtgcaacaattgttgtctctggtgatggccgctatttctcaaaagatgctgttcaggtgatttagaatactctccatctttgttatggttggcaacacctataggtgccggcaaaggggctaattaggtgggataagatcaagggggtcaaatcagggaggctagaactaaggagagccaagagtttaggagaaaatgttgctgtaatcctctttacaagcaattatagacaagtctaattctcacctcccttctctgttctctcacccccctgctccaactagataccagccaccacgggaaggaaacctgtggctgaatggtaggcaacctgtcactgttatcccctctggtcactaccccaaggttgtgacaatctcccttatatattttctaactgcattttgtaaaaactgatttggtagcagctattgtaagcttcagttcgaatactgatgcagtcttagtgttgttttctccctgtggtcgagtttttttacatggacttagtgttcatcagctaaatatgatgtggcaaaagatgtcattgcttttggttgttacagatcataacaaaaatggctgctgccaatggagtaagacgtgtttgggttggacaaaacagtctcatgtctactcctgctgtatctgctgtcatccgtgaaagagttggtgcagatgtaagttggatttctaataaaggacattctttagcaatatgaacatttacttgttactcctttacaccgtctaataaattatacctgactattggtttataaacacatggacaatagggatcaaaggctactggtgccttcatcttgacagcgagccataacccaggtggtcctaaggaggtacctattttactgctagtatctctgatgcaaatcaagtgctggtcatgctctatgttattgaatacatctcatcgaactttccaggacttcgggatcaaatacaacatgggaaatggtgggcctgctcctgaatctgttaccgacaagattttctctaatacaacgacaatctctgaatacctcatctctgaagacctaccagatgtatgattcaatgtcaacctgctaactatttactcaccgtttattttaacaggttagtggattatttattttctcctactttactgctgcaggttgatatttctgttgtcggtgtcaccagcttcagtggacccgaaggcccctttgatgtggatgtttttgactctagtgtagattacataaagttaatgaagtaagtaacttgttatttcgtgtttttttccattgaagtgagaatatcgatgctttatatcattacatgttacttggatcatgttatgttcacaagggaatctaatgatttaattactgacatcaattccaggacaatttttgacttcgaagcaataaaaaagctgctgacctccccaaagtttacattctggtattcatatatgactgcaaccatgttaccattgccgtaataatatgattattgcacatctaaaatctcactttgcactttcagttatgatgcactccatggtgttgcgggagcttatgccaaacacatctttgtggaagagcttggtgctgatgaaagctcactgttgaattgtgtcccgaaagtaatgagttcttctattgtcacaaatatctacttataatgtggctaggtattaattcatcacttatatttttgcaggaggactttggaggtggtcatccggatcctaaccttacctatgcaaaagagttggttgaacgcatgggtcttggaaagtcatcctcaaatgttgagcctcctgaatttggtgctgcagctgatggagatgctgaccgcaacatgattcttggtaaaaggtatgatctgtttgagtatcttattttctacaaaatttgttacagattctagaaaagcgcaaggaattcatgacactcccctttgccttgctatagattctttgtgacaccgtcggactctgttgccattatcgcagccaatgctgttcaatcaattccttactttgcttctggcctgaagggagttgccaggtaaaaagttctcctccatattgtgctgctaatatgacatctacatatattgttaccataaaaactcccactgtaggctccaggcggtaagcttcactgcagcctgcagtaaaaatgcatgtttccctctacatttgttttgcatctgtttaagcttggcagtgttcttctcaacaatgtgctaatgtgcttaacatatcagcaaatgaccatctggaacactatttctgctaatgtcagctttttcatcttccgcttgtatgcagctgtccggtcttagatgcatttgaaatttctctatgcactgaacactacttatgttattccattattgtaataacaggagcatgccaacatctgctgcccttgatgttgttgcaaagaatttgaaccttaagttctttgaggtaaaatattaaaatcttggtttgcaagatttttgaatgtcatataaatctgacataaaaaaaccatgtttcaggtgcctactggatggaagttttttgggaatttgatggatgctggaatgtgctcaatctgtggtgaagaaagctttggcactggtaatatatggcttgtggccttgtgggttgtggctatcatttctatacacacaacagtaaacatgtcagcaacctcttgtattgttatgttagatggataaattctgttaacatgtggtatataaatggggccaatcacttgtgttctgactgtgagctctctcgcccttctattgagttggtggtcatgaatccaacctatagtaaagtcaaaggacttgttagtatttggtataaacattggtgggatgtttacatgtactattgtggccagggtctgaccacattcgtgagaaggatggcatctgggctgtgcttgcatggctttcaattattgctttcaagaataaggacaaccttggaggagataagcttgtcactgttgaagatattgtccgtcagcactgggccacatatggtcgccattactacacacgctatgactatgaggtacttccttttttttgacatgccccttgcatatatgtaccacctgtttaagatatcattctaaatgcacaagtttttttttttgtgaagaatgttgatgcaggggctgctaaggagcttatggcaaacctagtaagcatgcagtcatcactttctgatgttaacaagtaagtgaattttttaaatgtaatataattagtacctccaaaaaaatatgctcgtactcctgtgtatatatatcaacttaagcaatcgcaaacttgaactttttagtaccacgtctaagttgtctctttgtcaggttggtcaaggagatccggtctgatgtttctgaagtagttgcagctgacgagtttgagtacaaggatcctgttgatggctctgtgtccaagcaccagggcatccgatacctctttggagatggttcacgactggtatgtaggcctgtgttgtttcggatatcctctattttgtcttgaactgagagtctgaaacaaagggagtggtgtgaaaaccttgttttataaagagaatacatttccgtttttgcaatattatcggtcagtaaaagataatctgagttggattcatccttggtcagatggatatatttgggatctctgactatatcatactggccacaactaatgctggtgagctaacatggatttttgacattctaggtgttccgcctctctggaaccggttctgttggtgccaccatccgtgtctacatcgagcagtacgagagggactcctctaagaccggcagggattcacaggacgcccttgctccgctggtaagatacctaccatgcggctatgccctttcgattcacagagcataacataccttgggcttctgtgaaactgcgtttactaatttgtgtctcatcccaggttgatgttgcgctcaagctctccaagatgcaagagtacactggacgctctgcccccaccgtcatcacataaattttgaagagtgttttagaatgagttgaggcgcttacacaaactttcattccggcctcttgttccatagtttttcttgcatgttacatctcaccgatgaataaaatgtatgtatcagacttgtctcgtttttttgcccatccaagcagcaaattagccgctggcacagcatgcggtaataatcttgtcacagtgctgtaattgggagcgtttttcttgttagaagtgtttctggtttgtttgagcatttgcggatcgatttttctttctgaagagtatataaacattttactcacctgtatacactgtcgcagtgtcactaatgt

SEQ ID NO: 11 - DNA, 10841 bp, Zea mays
  source 1..10841: mol_type = genomic DNA
gagagagagactctcccggctcccctgcctgcgcgccgtctcctccaggctccagcgcctcgcggccaccccgtacgtaccacggcacggctgcacggccgatggcgtaccccgccgcgccgcacacgagcgcggccttcctcgctagcttcgcgacgccgctccccgcgcgccgcgcctcgcccccgacccggcgccgcgcggtgtcggtcgccgccatcgccaccaccaccacctgcgtcttcgccgacgccccccgtgggatcaaggtcagtcgcgctcgcgcattgccgtgaggtccgcgtgcttccgacttccaacaattggacatgctagccgccgccaagggcctaggccacgctggtgtcaagcgtggcgtgatctgtgttgcgttcgcgtgccgtgccgttccgtggtagggcgggtcatgggtcaatcacattttcgggttgtcacctgtggcaggaggagcaggcggactcgggcgcccaggcggcggcggctgcggctgcgcggagggacgtcagcccggacaccgtggcctccatcatcctcggcggtggcgccggcacgcgcctcttcccgctcacgcgtacaagggccaagcccgcggtaataaaatcgcctcagcacctccctgtttctagactttgtctccatcgctcataagtcacatttcgtcattcatcaatctcttgtacggcattactacacaacttgggattagcggttgtagattcccgttgcaatccgtgccatcgactgagctacggggtagttactgtaatagtgattcagtcgccgtctctcgctctcaggtgcccgtcgggggatgctacaggctcatcgacatcccgatgagcaactgcataaacagcaagatcaacaagatctacgtcctcacccagttcaattcccagtctctcaatcgccacatcgcaaggacatacaactttggtgagggagttggattcagtggcgggtctgtggaggtacactcttcggtataagcaggctcgattgcggaataccattgcgatggactcctgatcttgttttctgcctgtttattttttgctcattggataggtgctggcagctacccaaacagctggggaatcaggaaagaaatggttccagggaacagcagatgctgtcaggcagttcctctggctttttgaggttcttcctgggccatgtgtaatactctacctttcagagtatgctatcactatgtccaattaactttttgcctgatgttattctgtgaaggatgcaagactcaaatgtatagaaaacatactgattctgtctggtgatcatctgtatcggatggattacatggattttgtgcaggtctcctatctgctgcaagttgatgttgtaatttcaagtttcagtcttatttttcattgcttgacaaaggtatattataatattgttttgcagaagcatgttgactctggtgctgatatttcagttgcgtgtgttccaatggatgaaaggtcatccagctttgtcccttttaaatttgttccttgccttttcttggtaattaaactagtattcagttggctaacaaagcaatatgccaatatgatgatatcttagctacctatactgtatgcactgtttattcagtagtgcatatttgtttatgactatcatcaatgttcatgctggaaagcttcagatatctcatgctttgcattttataagaactagaatcactttgtttactgttgtcatatcccacgctaacatattcctatcttcttattttgtttttttatctcatatatacatgcagtcgggcatctgattttggattgatgaaggccgacaggaatgggcacatcactgatttccttgaaaagcccaagggtgcagatttggaatcaatggtatggtacactccctctcactgaccccaaagatgaacaactacttaatgatttattcagacaagtatagattgactgcatattgcctttcttttgcataattagtatttttgtcatgggcttccttttatgttgcatcatcttctaaattatttgagttgaatcctatgtctccctaacttcatttcaaatggtatccttggatttatttactttgtggaaagatttttaaatactctattgtacttgttaatgaagcaagtggacatgggattgtttggattatccccagagtttgcaagtacgtacaaatacatggcttcaatgggaatatatgtattcaaggcagatgttctcagaaagcttctgaggtaagaattagaacaaacttgctaagtaaggcttttgttggttatttttaaaaccttttacgtgaaagaacaatgtattgatgacatttcgtgttcacagtttgtatataacgatacatctagctttcttttgattaagccttttgtggttcccaacaaacgtgaatttaaccacaagttacaaaattgttacctcttttgacttgtatcttgtaagacaggaattatgacatcttgcaatctccatgcaccttcattcacattatgttgcagctggtggtgacgacacttatgttctttctttgttgtgcatatttattagaggccattatcctacagctaatgattttggcttagaagtgattccaatggctgcaaaagattatgatgtgcaggtatttacactctacattcgtcaaaaatccaaaatgaacttatgttaccttcttttagttgtcatgaacacgtggtctgaaggatctgttcttctgttggctccaggcttatttgtttgatggttactgggaagatattggaacaataaagtctttctttgaagcaaaccttgctctcactgatcaggtttacattttgattaacattgtttctggcttaatctttccgagcttggataatagtttcattttttattttgtagtctcccaacttctatttttatgatcccgtgaagcctatttttacatctcctagatttttacccccaaccaaggtagaaaactgcaaggtcagaatctcttattacctatacattgtttcgtttataatgcattacctggcagccttgcttgatttatctgtttttgtgcaggttttgaactctatagtttcacatggctgctttctaactgagtgcagtgttgagcactctgtcattggtatccgctcaaggttagaaccgggagtgcagctcaaggtaaattattgagtgtttgtgagttatatagttgcaaaggaaatctaaaattatcctggtggggtgggagttaggtggggaaaatgttactctttgatattcagtttgcatggttgatattcataactttattaaggccttgttcgattgtttcaattccatgtggattggagtgtattaaaatagattggaatggattttaacttgctatggatttaaaccgactcaataccatctaatccatatggattaatgttaaaacgaacaagccctaagggagatctaatgaccacaattgtaattaaacatatgaaatgctaaataaaacatcacatgatgtagctcaatccattgtcacgtccatcagactcttactctgtagtgcccatttatttatgtttgttttctgcaatatacacatttgtgacatcactgaaggaagtagagaatgcgcataatgtgtgggatgatttattgcctgagtcctcgagctgtttatataagagtacatggcttggagggcaaggcgcctctcccagagataaggaaggctaccatgggattacaatcaatcctaaactatccatatccggtgttgcctaatatactctaagaccccccctgtcacagtgggagcggtgcagacgatgagactgtagaagaagcagtcgagctgacaccccccgcagtcgtaatggtcgatgcgacgcagatgctgtggctggagtggaagccgacgaggttgcccaagcaaggcggggtagccctttgtgccgatgtcgaagtagccaagcgtgaggatgagtagccgtgtcgaggtgctgtgcgtcgggtagctgtgatcgatgtagctgtcgaggatgccaaagacaacaagccacacaagtcgcgggtgcatcacgcgggaaggcccgtagcgcgccaggaagcgccagagtgatggtggcgtaggaggacgccatagacgaagatggcgatgtagtcgaggcagtcatagaggaagtagtcgatgccggggacgatgctgtcaaaccgccatggacgaggcgtgctccgggtttgccacgcctggaaacacgtcgtagacgaatgcacgtgccagtgttgccagtaccgccgtactaggtagaaggccccaaccaagtgtcagcgtctgaacagagaagacctccacgatggttgtagtgccgtatggcgggagtgccgtcgtagctcacagttgttagagcggagtagacgaaatagtcgaggatgagacgacgacgctggcaacaaggatgtgctggacgaaggcgaagaggtaggcgtggtgacgcatcgaggatcctggagtcgcaacggtgagagttacgacacacatgaagagactcgatggcgccagaggagcgcgacgaaggtacttgtcggcgaagaactcgaggacaaatgtggtgtagaccagcgacgagtatgtcgacgcagcttacaggtgtcaaacagctcgtagaccgtgcgcaggagacgcacaccgatggaatcgacgtgtagatgcaacgatgaggatgccgacagacaacgatgctgctgcccgaggaggcgacacaacgacggccctcttgcttggcgaagcatcgcgcgatggacgataaacgtctcgggagggtggtttgcccgcgatcggttgatcaggccgatgacgtgcgaggcggagacgatgatggtggagcctcccgatcggtggtggcgacgacgagccggcatagagccatttgcaggcgaagaggcgcgacacaggcaagcgttccggttacgccgcatgccacgccagttgcaccgtcgaagaagatgaggaagccgatgtcgaggatgtgcgagtcgactggcggtgggcgacgcaaacccgatctctgatcggggaaaaaaggaaaagatacacacaacaacaacatatctagggacatcaccgaagtaccgatagccagtccaccaatcctctgttcccctagcgtagagaatcaagtcacggagaaagatagcagagcgggtgactagcccccatcctcctgtcatcattctctagaatagggaagagagaaaaggcatatccataccatcactgtagcgaccaagggggcggcgcgacccgtcatcacagtgacagggggaagggcgtagcccgcgatcaccatcgcggcgcgacctggcggcgtggctcggcccggtggtgaggagcccagcatcgctccatggcgaggggcggctcggtggcgaggtgccacgacatagctccgaggcgaggggaggcccgacggcgcggccgagtccgcggcttgatcggccgagtccgcgtgggtgaaggcctagccggcggcgcggaggggcacgactcgaccaagggtggacgcaggcacacagagggacacaccagtggtggagcagcctctgcagatccgagctgcctccccgcggccgtcaccacgacggcgagcggaggggaagctaggacgcggtggcctggccacagtcgtcaccatgacgacgtgtgaacaggacggagcgaggcagagctagccggcgacggaggagacaaccaccacaatgcggatcaggtgaaagtcccgatctgctagtgcttttgacacaacgacgacgggtggcggggcgacgtgaagtacgcgacgactggcgcagcaaatcagagggatcggctgcgcatcctacgagggcgctgcccccagaggagatgatctccctgggggcggcgcggaggcggtagtggaagactaggctaggatttagaaaccctaactcgtgataccatgaaggaaggagagaatgcatataatgtgtgggatgatttattgcttgagccttcgggctgtttatataggcttggagtgcaagacacctctcctagagataaggaatgctatcatgggattacaatcaatcctaaactatccatatccggtgttgcctaatatactctaacaatcaccatgcatcattgagcatgtatgagctcttttgtttgtttagttttgtgccgatttcgtacccttttgttagttttgaattttaaagcaaaatgtgcatgggtatttaatcgtcaactgtactctgcaatgcgagtagttatccagcattgataacacaattttctgttcttgctgaatattgtggattgcttgtttattttcaagtttattaatgaacaacaaaatggtttaggacaccatgatgatgggggcagattactaccagactgaagctgagagattatctgagctgtctgttggaaaagtcccagtcggtgttggagagaacactaaaataaggttattgtctctttctatattaatgtgtttgcctaagtattatgcatgtttcattgacgtagagttttgtcacacaggaactgtatcattgataaaaatgctcgaattggaaagaatgtagtcattatgaactctgaggtatgaaacccctctttataaattttatggaaggctttgaagtccatacatttgatcctattcaatatctgctaggctttgaagtccatacatttgatcctattcaatatctgctagctcagttccttagccatttactgataaaataaaatcactctgtaagaatgagcatatcatgctacataagataactcggtaaggatgagcatgaacgtgcttgcaataatgaatcagtggttgattcttaatttgatgaccaactataaatgacaaagaatgccagctttttcttattttggcatagaaatagccttatctcttaaaaagtcattgctagaatgagatgaccttaatctactttgctaaaaaaagatggccaggaaaacccattgtataagggtccaatatgacatataatgagtgtattacattctgcaaatgttatttttgttcatattatgaaatttggcctcctttggattgagggaaaattccccgtttcctgtgaaaatgaactgattcctgtcaaattcctatatgattcctgcgttccaaaggaggcccttgtttgtcatttttccagagtaagatgttgagctgagaatatccccttatgaatgcagaatgtgcaagaagcagacaggccagctgaaggatattacattcgttctgggataactgtggtgctcaagaatgcagtaattctgaatggtacaaaaatttagaagattatagaattatctgcaggtaagctaagtgtgatgcttaagaatatggaactaatattattgcacatgataatttgatttacaaaaaattttataaagttagagaatgaatgttctgtgtacaatgcagaaatattgacaaataaacaagttaagcctatacataatgaataaacgaagaatgtccagatttgtctagtgttatcaccaaatcaatttctactataaagttaggtagtgaagaaacactctgtaaacagtgtactaattgctgacatgtaaagaatgaaaaggacgagttaataatattcagagctgtgtaatgtaggtcccactttaattgcctacgtctaagggtattatgcctgcctgtgcaatatttccatacaccctaggcaggcagaagcttatcaatattgcttgcagcccgtcctactccttgagaagttgagatcaaccttttccactgcctaaatctcttggaacttgtacccttatgccctcctttcttcttttcttgtcagaggcagaattctgagaaccttaacatattcgttttctaggataggattatctgctgtcatcagactcaccatttcttttgaaagtgctgttctgtttctagggcttgttcccaaatcgatgaggtcgaggaatgcggtatggtacttgtgagaggcttttacatgatggggtggaaaatgatcccatgtatgttgagggcagggttcgatttcccggtagagaagaccgagaaaccggtaaatttaccgtttccggtcgagtacgagatgcatttttcagtcctattttctatgtgtttcgattccaaattttaaaatacgaaagtcaacatttttggtgaaaactaaattttggaacagtaaaaacaaaaatgtaaaccacagttgaggtgggatgctgatgctaaagccagcccacggtctgttagggtcggttttctgaacccacaaccaatggattctcaaagaccaacccaggacttccaagttatgacgttggagattaatctggagaagctggactcctctccactgcaaattcttcctttggctcgccaagaacaaccggtgttagactgcggagcgacttgccaagcgtggtctcgcccacccagctgcctgccccttgtgtgaccaggctgatgagacccttcagcacattctcatcggttgcgtcttgtctagacagatctgggccttgattttgcagtccttgcagctggctgaaatttcccccactctggctgactcgggtttcttctcctggtgggcgcgatctgtcaagtcggtgccaaagaattttcgaaaagggctgagcactctctgtattctggtagcctgggaaatctggaaattcaggaacttgtgtgtgtttgagggtgtgcagcctaatgtccagcttcttctccttaggataggaagcgagggcatcctttggtgtgctgcgggagcttttaggttgcttgagctccttaagggttgatttggtgacaaggggatcacgggaggattagagggggttgagggggaaatgaactagtttccccctcaatccctccaatccccctatgatccctgatcaccaaatcaaccctaaggccctgttcgttttgtgctcatctatagggattgagggggattaaatcccctacaagtcaaaatcatctccaatcccttccaatccccctcaatccctatggttttgggatcaaacgaacatggcctaatagggcgctgatcccagctagctagctaggcgctggtggtcttggttgttctttttgttttcagttactttgtaaaacctgctaagggcctgtctgtacttgggttgggtttcttgacctttcccgctgtttttcttccttaaatgaaatgacacagctctcttccttaaatgaaatgacacgcaactctcctgcgtcgttcgagaaaaaagtaaaccacagttgagggtcgatgcatttggacctgtttttatgattatatcatcctcatatcactcatgctaagtacggcagtggaaagatctgcatataataaatccttactatctactaaacatcacgtgaacaaaaaaaggttggcagggggaagaaccgtttcttgtatgtattgggaacatgctgaacaaatgtattgacactcaagggtttctcctagattgagctaccctggagccaatgtaccatgatagtagcatttttgccactaaggccattcactgtaaatttcaacgaaatatacatgttccttgactggtctgctagggttaatttagcactagcttcagacctgttcatactgtagggtatttttctcgaacacgaaggagagttgcgtatcattatatattaagaagtagcaaattccaaaatagacccaaaaacaaaggtacaaagaggccaattcaaggcggccaaaccttacaactactcaagctagatagagctcaaccctgaggcaccggccttacaccttacaccagatgtcggcctcatccttgatacattgcacaatagactgatggctgggagtagcctcataaaaaaatgcaagcgtttatgtgcttccagatccaccaagtcaccaacatcaccagagaattgaaaccttttctagactgtttctgaacattcatttcgttgctgccccaccaatcttgaaagttgtgctgaggtcctgatactgtagggtatgtgccagtgcatttgaggatacgcacaaattggagttagcagtacatgcttggagatgatgtagtagttgacacatgcaaatgctttggaataactttggtacctgctacctacagttcctggagtaaagaaaaatggacttcaacagcttatatctggttgaaacatgactcaggaagtcatgatagtgctaattatctggttttccgattccaatcctagaaactattttgattattagatgcgtaccatgtggttgctgctttgaaattctatatgccaccgagtcataaatccatgtctgtaaattgcagaataccaatccctcagtgaagagcacaccattgaagcccacagcagagttctacgacgcagatgcagtcaataaaaccccaatagcacaaaagattaggagactgtcgatgcagctaaagaagtgtggatataggcatggagcacctgcaacacgccaacggtacggccaaaccatgagcatgaggccatttcacgtcctttcataagcctcggctagtgccggcgcgcgcagggtatcttttgttccttttttctagcagtaatttacgtgaagtgtctgtacatgtagagggagagatagggtacaagtacgagagagcatgttatagtacctactcttattatttgtttatttattgttgtgccgaagaacgacccgtgcatctcatgtgccaagtccatgcctaattgc

SEQ ID NO: 12 - DNA, 3175 bp, Zea mays
  source 1..3175: mol_type = genomic DNA
atcccacccctcgcccaatgcgtagttgtatctgcatctccaacaccaccagcagaggttccagttagttttttttagattagaaaaatcgagtccaacagactcgccgcactgcgcgggatcgacctcgtcgcaactcccgtgtgcgccgccaataatggagacccgggacacggatgcgacgccgctcccctactcgtacacgccgctgccggccgccgacgccgcgtcggccgaggtctccggcaccggcaggacgcggagcaggcggcggcccctctgcgcggcggcgctcgtgctctccgccgcgctgctcctagccgtggccgcgctcgtcggcgtcggtagccggcccggcgcggtggggatgacagagtcggcggcctcgtcgccgacgccgagcaggagcaggggccccgaggccggcgtgtccgagaagacgtccggcgcgtctgacgacggcggcaggctccgtggagccggcgggaacgccttcccgtggagcaatgcgatgctgcagtggcagcgcacgggattccacttccagccgcagaagaactggatgaacgaccccaatggtacgttacgatcctctctgtctctctctctctctctctgctgctgctgcgctgctgcatttggtttgggttgcatcccaactcaacatccccttccaaacgactatggattagatcgatttgaaccatatgaacgaattaattgtccacgcatttgattcctttcacgtgcatttcatccaacgaggcgctgggacccacacgcatggacgaattattcagcgggccccttttttctcttcacttttcccttttttctcttcacttttcccttttttttttctttctcatccacatacaaattccagatacccccgggccctccgggattccacccaaccaccaggcctaccgttgtgtattcttcttttgtttttctgcccttctcgtgcctgacttgtttggtccaagtcgtcgtcgcttcgtaataataataataaattgtaaattatacgcttgcaggccccgtgtactacaagggctggtaccacctcttctaccagtacaaccctgacggcgccatctggggcaacaagatcgcgtggggccacgccgtgtcccgcgacctgatccactggcgccacctcccgctggccatggtgcccgaccagtggtacgacaccaacggcgtgtggacggggtccgccaccacgctccccgacggccgcctcgccatgctctacacgggctccaccaacgcctccgtccaggtgcagtgcctggccgtgcccgccgacgacgccgacccgctgctcaccaactggaccaagtacgagggcaacccggtgctgtacccgcccccgggcatcgggcccaaggacttccgcgaccccaccacggcctggatcgacccctcggacggcgcatggcgcgtcgtcatcggctccaaggacgacgacggccacgcgggcatcgccgtcgtctaccgcaccacggacctggtgcacttcgagctcctcccgggcctgctccaccgcgtcgacggcaccggcatgtgggagtgcatcgacttctaccccgtcgccacacgaggcagggcgtcggccaacggcgtcgacatgtccgacgccatcgccagcaacggcgccgtcgccggggacgtcctgcacgtcatgaaggccagcatggacgacgaccgccacgactactacgcgctggggaggtacgacgcggccgccaacgcctggacgccgatcgacgccggcagggacgtcggcatcggcctgcgctacgactggggcaagttctacgcgtccaagacgttctacgacccggccaagcgccgccgcgtgctgtggggatgggttggcgagacagactcggagcgcgcggacgtgtccaagggatgggcgtcgctgcaggtactttttgcttcgctagctaacacgctaccatcacactagctgtcgttggttgctgtttggtacctacttactagctagctgctgtattggattagactagtggttgatattcatgtgccaccatgtgcctttgcagcactactggcttagttattagtacagacaacaaggaaaagggcggcattgtctgcttgtataataattgaataattgcattgcattgcagggtatcccccggacggtgctcctggacaccaagacgggtagcaacctgctgcagtggcccgtggaggaggtggagacgctgcgcaccaactccaccgacctcagcggcatcaccatcgactacggctccgtgttcccgctcaacctccgccgcgccacccagctggacatcgaggcggagttccagctggaccgccgcgccgtcatgtcgctcaacgaggcggacgtgggctacaactgcagcaccagcgggggcgccgccggccgcggcgcgctggggcccttcggcctgctcgtcctcgccgaccgccgcctccgccgcgagcagacggccgtctacttctacgtggccaagggcctggacggctccctcgccacgcacttctgccaggacgagtcccgctcctccagcgccaccgacatcgtcaagcgcgtcgtcggcagcgccgtccccgtgctggaggacgaggccacgctctcgctccgggtgctcgtcgaccactccatcgtcgagagcttcgcgcagggcgggaggtccaccgccacatcgcgcgtctaccccaccgaggccatctacgccaacgccggcgtcttcctcttcaacaacgccaccgccgcgcgggtcacggccacgaagctcgtcgtccacgagatggactcgtcatacaaccacgactacatggcgccggtggcagacatctgatcgatgcaatgcaaccacgccactctgcaccgcaacaaattttccaacacacataaatataccaagcaattcccatctgtcaaattttcttatttccctgccatgattttggtctctatttttcctgctggctactacttatcttccagcactagtaatgtacgtactggctagtaactagggtatccagtgggaagacgtgtagcctctagttgtacatgcatgtatgatgctgtgtagcaccgcatcatgcacaccacaccaatgcaaatgagttataaatgtttttaagctccaaattaaaa

SEQ ID NO: 13 - DNA, 6472 bp, Zea mays
  source 1..6472: mol_type = genomic DNA
ggctgtcgtctcgtcccactcccactcccacactcccactccgacgcttgcgccgcgagaggagaaagtgccggagacagaagcgaggagagagagagagagaagagaagcggggggctcgcagaggcgtagctcgcgatccgggcgccgagctggtgcgtacttggtttggtcgcgcggctcggtcgggaggctagctagctagggctcgcggcgggcgggatggcgaagggcggtgcggtcgtggggacggctgtggtggtgtgcgccgcggctgccgcggcggtgggggtagcggtggtgctgtcgcgcaggaggagtcggcggcgggaggcggaggacgagaggaagaggaaggcggccgcggtaatcgaggaggtggagcagcggctctccacgcccaccgcgctgctccggggcatcgcggacgccatggtcgaggagatggagcgcggccttcgcgctgagccccacgcgccgctcaagatgctcatcagctacgtcgacaacctcccaaccgggtaaggccaggtcgggagccccacccaccacgcgagtccccgtgcatgtggctgcacgttccacagcccacacagtgtccgtttctagcttgcttgctcttgcgctgcgcggccttccgatcgatccgctgggctatccaggtttcgattaggtggaaatgtcggtctgtcccagattggatacggcgctttcgagcggagccaggcgggcgatgggtgtccgagtccgtccgatcttgttgattctggcaggaaggggaggggagtccttgtttctctcatcgtctgtatagagctctggttcatggctgggcaccttcgcaatacgtacagtcttgtcttttaacaccgcaacgttggcgggatgctggtgtgaccgtgtgattcatatttttttctttttttgcttcgcatgttgttcatcaatgcgttcggtctccaatcttcagtttgcgtgtaaaatccgatttgggttgccgaggtgactataaggtcaagggttatgggtttgatgtttaatttgtcccagcctcaccctcacagatcattccaaaactacaaatgtcttcttttgataaatataaacgaagctttcgtgttcctggctgtacctacactacatctacatgtgggactcactttcacggaggaccatgatgtcgtcctttctacgtcatggctaatgtctaaagtctcatttacaccaccacctttccggtactgcctctgtcaccaaatgtttggcgctactgaaacatgtaaacgtagtcaacctttgtaatcgtggacccaagatatttttctacttagttattactgaatcacacacacacacacacacacacacaaaagcctgtattgttggatctgtcataaatataatatagttatgacgattcttcccaagagatattgtatgttgcaataaaaaacaaatacagctagctacatttacagcttgtttggatgctcaggttttgacctcaatccatgtctgttggaggcatccttatagagcataatttgctgatgaaacatgtggctcttgcttcgtgaatccttcctttatggcctcatctgccgtcactatcttgtttattgatgtgtggcatcctgcagggatgaacatggtctgttttatgcactggaccttggcgggaccaactttcgtgtcatccgggtccaacttggcggaagggacagacgtgtcgtgaagcaacagtatgaagaggtctccattccgcctcatcttatggttggaacttctacggtgagtgcttgaatttcgctaacaaaagaagtatgcatgtttcctctcaaagctatgccaattaataagcaaaaaggggaaacacattgccaccattcccattatgacaaagttgccccaacataacccccctaccttttgtaagttaccaaccattaccctcatgaaaaagaccaacacaaaatacccttttacatatttataaattacccactattgccattgaaaaatcaaccgatgattcccccatgcatacttctctattattccacttatgccattattttaaaaatgaaatgaaaaaaataatacaataaagtttaggtaatgtataagtttgtgtaaacataatgcatttaagaggtcagtgttgcaactcacctggcagcattgctaattagtcactcactagaaacaaaagtattgttcaatccaaagaagcatcaaccttgcctttttagctaaatgtcgctttgatgatttacttgcttggttgctcccatgagtgatgctttccctttttgactattttcgtcacaagcatgacccttctgcactcaacccacacacaagctgccttttcgggcttttgatttttctggtcatgtatttctccttgatctttacttgcaaaccattgttgctatatagacaaaaaaaaatatgtgaacaaattcatatgtacaaaagttatgcacaaaaaattgtataaaacaaaacttcataattatacaaaactatgtcggaatctttttttcccggaaaactatgtatgaaacttaaatacataaattttgttttttaacatatatgatagacaacatgcacacttagattcttgacactagttctgcaattgcaggaaccactccactagtttcatgtctcctgtatcagcatgctttaatgacatttctcaaagcatgattacaggaactatttgatttcattgctgctgagttggaaaaatttgtgcggactgaaggagaagatttccacctaccagatagcaagcagagggaactgggtttcaccttttctttcccagtgcaccaaacatctatatcatcggggactctaattaagtggaccaaaggattttgcatcaatggcacggtaaaaacaagaatacatttgttcctactttgttgactatattacttatacttatttatattaccagtgccttggccagaagctgactatgctgccaagatatggattctctctaattactacacatttttcttgaccataccatgtgtcaatttagctagtgcatttcttttttttttaccgaaaacatcttattaaattggcaatggagtagactttacctattcatgctgttttttttacttgtggaacttctggcacaaataactgttttctttcattgagaaaatggatatatttttttaatatacatgatgtttgtgccaactgtgaagtgtcctacattaaaacacagtattgattttgttgattcctgtgagcaggttggagaagatgttgtggctgaattgagtagggccatggaaaggcagggtcttgatatgaaagttgcagctctggtaattaaattagtttgttcactatttatcatatgaagtactcacatagacacatactatagaatttgccatttatttatgccttctgatgttgctttgagtttcttgctatgttttatcttgacgtccagcaaaggcccattgaacatattagaagtaaatacaacatgttaactgataggtaacatttttttacaggttaatgatactgtaggcacattggctggtgggagatatgctgataatgatgttgttgctgctgtaatattgggcactggcacaaatgcagcttatgtggaacatgcaaatgtgattcctaaatggaccgggctgctacctagatcagggaacatggttagtgtttaacttccatcgagtgagtggaattttaatagtctttggaggatgaactagctcaattgatgcgaagtgtggttgtgcggcccaccactcaatgcaaattcccttcaactcgaatttgggttcatatttcttcttagttacaatgccacctagttcctactatgttggtcaagtttttattttaaatgatcctttgcaccttgctaaattgtatccgtccaacataagtttgccttctgtaattagtatcttaatatttctcaccagttaacaatttaaaatttggttcttttgaaatgctacaggtaatcaacatggagtggggaaacttcagatcagataaacttccaaggtcggagtatgataaatccttagacttcgaaagtttgaaccctggtgagcaggtatgcctatttaatctatgcagctgtttattttggtattcattttcttcctaagtaagtcgcacattttatacattggatcaaatccaatcagattatacacctatctgcatacagattcaatcatccaaatgtcccaaatgttgactcagattttaggggggcaaaagtgactgacggatatcaaaaccattaatcaattgttctaagaactatatttggtggtggagtcgtgggtgcatgactccacccagcagggttcaaatcctggctatgcacctatttagatgtttgtggagtaaaatatttgggtgttgaacttgaacataacctatgtaccttttgtcagatatatgaaaagatgatttctggaatgtatcttggagaaattgtccggaggatcctgctgaaactggctcatgatgcttcattgtttggggatgttgttcctccgaaactggaacagctatttatactgaggtttactttcacgttccatcctctaacaatattgcttttcttatatttgtagaatacacaaaaatccaaaatcaacttatataaaggtgcacacatatttgcacaacctagagtccaaacattttgatagattgtggatgtatttgtgcagtttatactatgaattctaccatacataacccacatagaggttccagattggaaactgaagtcgtaagacttagggctttgataatgttcctctcactttctttaaaatgtttatttacatggccaatcattaaattaaactcatgtaatttggtgcaatctccctacttattcagtctttttaagcatgtaaggaaacaggtgtctctctctccaccctagagtctaatcagcaattttgcaaatccacctgtttggggtgatgttaagatggctgagctgcattaagtattaccgtcccttgtagtccccaacatctaagggtgcgtttgactgtagtgacagaacaggatgggataggacgatccctcaaataaggttgtttgtttcagggttaggagttgggacagtattatcacagtgttgtctccagttgtccctcaaaattgaagggacgagagaggacgccaggggcgtccctgtcctgactgaccaacacatcctaaaattctcatctgactcccagagtactgaaaaaaggaatcaaaattctccttccagtgcccatctagacctttaaaatcacacccataggctaaacctaaaaagagactgaagaagtgaaaatgatagtcgtgaatttcttagtctaaatcttgttgtatgagcacatgctccaagttgtttatcgtagattacttaaaaaggttgtccaacaaacttgtgaagcagaattgtaggtctcgtgtttctgaatgaagttgcgattgtgatatggcaggacgccagatatgtcagccatgcaccatgacacctcacatgatctcaaacacctgggagctaagctgaaggacattcttggggtaatggtgatacacatgttcaaaaaagagggtattttgctagatataatcttcaatgacgattggttccttatttgctatgtttctgcttattctaggttcctgatacttctctggaagcaagatacatcactcttcatgtgtgcgaccttgtcgcagagagaggtgcacgcctggctgctgctggtatatatggtattctgaagaagctgggtaaagacaaattgctaggtgactttagccaacaaaggactgtagttgctattgacggtggcctatacgagcactacaagaagttcagtgcctgcctagaggcgacgctcacagacctgctcggcgaggaggttgcctcatcggttgttgtcaagttggccaacgacggctcaggaattggagctgcacttcttgctgcctcgcactcccagtatgctgaagctgcatagttctaggagctcgggggtcctagtgtaacctttttttttgtttcccaccatttcccccttcaatcgagaagcccccctcgtgcgattcgcatctctctattttctttcctcagcagcgggatatagctaggatggtgccattgagcttggagtacattatcactagcaacaagcattagcagcggcaggaaagaagtggaagcggaggcctgaataagccgttccattcctgccgattctttttcttcccaatcccttgaagtttgtattatgaggcgatatcacaaaacagccagttcttatctgtgtgatctcgtctttcaaaataaggagagaggtgggggaccaaatcatacagtctttactcattttttattgaatcgtgcagtgcttctgcctttatt

SEQ ID NO: 14 - DNA, 3990 bp, Zea mays
  source 1..3990: mol_type = genomic DNA
gttttgggcgcgagcgctggcgtgcgggtcagtcgctggtgcgcagtgccggggggaacgggtatcgtggggggcgcgggcggaggagagcgtggcgagggccgagagcagcgcgcggccgggtcacgcaacgcgccccacgtactgccctccccctccgcgcgcgctagaaataccgaggcctggaccggggggggccccgtcacatccatccatcgaccgatcgatcgccacagccaacaccacccgccgaggcgacgcgacagccgccaggaggaaggaataaactcactgccagccagtgaagggggagaagtgtactgctccgtccaccagtgcgcgcaccgcccggcagggctgctcatctcgtcgacgaccaggttccgttccgatcctgtccttgagtttcgtccagatcctggcgtgtatctacgtgtttgatgatccaggttcatcgaatctaaatctgtccgtgcacatgtcttctctctctctgtctgtctgctatgcagtggattaatcggcatggcggctctagccacgtcgcagctcgtcgcaacgcgcgccggcctgggcgtcccggacgcgtccacgttccgccgcggcgccgcgcagggcctgagggggggccggacggcgtcggcggcggacacgctcagcatgcggaccagcgcgcgcgcggcgcccaggctccagcaccagcagcagcagcaggcgcgccgcggggccaggttcccgtcgctcgtcgtgtgcgccagcgccggcatgaacgtcgtcttcgtcggcgccgagatggcgccgtggagcaagaccggcggcctcggcgacgtcctcggcggcctgccgccggccatggccgtaagcgcgcgcaccgagacatgcatccgttggatcgcgtcttcttcgtgctcttgccgcgtgcatgatgcatgtgtttcctcctggctcgtgtatgtgactgacgtgtgtgttcgggcatgcaatgcatgcaggcgaatgggcaccgtgtcatggtcgtctctccccgctacgaccagtacaaggacgcctgggacaccagcgtcgtgtccgaggtacggccaccgagatcagattcagatcacacatcacagtcacacacaccgtcatatgaacctttctctgctctgatgcctgcagatcaagatgggagacaggtacgagacggtcaggttcttccactgctacaagcgcggagtggaccgcgtgttcgttgaccacccactgttcctggagagggtgagatgagatctgatcactcgatacgcaattaccaccccattgtaagcagttacagtgagcctttttttttgcccccgcctggtcgctggtttcaggtttggggaaagaccgaggagaagatctacgggcctgacgctggaacggactacagggacaaccagctgcggttcagcctgctatgccaggtcaggatggcttggtactacaacttcatatcatctgtatgcagcagtatacactgatgagaaatgcatgctgttctgcaggcagcacttgaagctccaaggatcctgagcctcaacaacaacccatacttctccggaccatacggtaagagttgcagtcttcgtatatatatctgttgagctcgagaatcttcacaggaaacggcccatcagacggactgtcattttatactgactactgctgctgctcttcgtccatccatccatacaaggggaggacgtcgtgttcgtctgcaacgactggcacaccggccctctctcgtgctacctcaagagcaactaccagtcccacggcatctacagggacgcaaaggttgccttctctgaactgaacaacgccgtcttcgttctccatgctcgtatacctcgtctggtggtggtgcttctctgagaaactgaaactgactgcatgtctgtctgaccatcttcacgtactacctaccagaccgctttctgcatccacaacatctcctaccagggccggttcgccttctccgactacccggagctgaacctcccggagagattcaagtcgtccttcgatttcatcgacgggtctgttttcctgcgtgcatgtgaacattcatgaacggtaacccacaactgttcgcgtcctgctggttcattatctgacctggattgcattgcagctacgagaagcccgtggaaggccggaagatcaactggatgaaggccgggatcctcgaggccgacagggtcctcaccgtcagcccctactacgccgaggagctcatctccggcatcgccaggggctgcgagctcgacaacatcatgcgcctcaccggcatcaccggcatcgtcaacggcatggacgtcagcgagtgggaccccagcagggacaagtacatcgccgtgaagtacgacgtgtcgacggtgagctggctagctagctgattctgctgcctggtcctcctgctcatgctggttcggttctgacgcggcaagtgtacgtacgtgcgtgcgacggtggtgtggtgtccggttcaggccgtggaggccaaggcgctgaacaaggaggcgctgcaggcggaggtcgggctcccggtggaccggaacatcccgctggtggcgttcatcggcaggctggaagagcagaagggacccgacgtcatggcggccgccatcccgcagctcatggagatggtggaggacgtgcagatcgttctgctggtacgtgtgcgccgcccgccacccggctactacatgcgtgtatcgttctactggaacatacgtgtgagcaacgcgatggataatgctgcagggcacgggcaagaagaagttcgagcgcatgctcatgagcgccgaggagaagttcccaggcaaggtgcgcgccgtggtcaagttcaacgcggcgctggcgcaccacatcatggccggcgccgacgtgctcgccgtcaccagccgcttcgagccctgcggcctcatccagctgcaggggatgcgatacggaacggtacgagagagaaaaaaaaacatcctgaatcctgacgagagggacagagacagattgattatgaatgcttcatcgatttgaattgattgatcgatgtctcccgctgcgactcttgcagccctgcgcctgcgcgtccaccggtggactcgtcgacaccatcatcgaaggcaagaccgggttccacatgggccgcctcagcgtcgacgtaagcctagctctgccatgatctttcttctttctgtatgtatgtatgtatgaatcagcaccgccgttcttgtttcgtcgtcctctcttcccagtgcaacgtcgtggagccggcggacgtcaagaaggtggccaccaccttgcagcgcgccatcaaggtggtcggcacgccggcgtacgaggagatggtgaggaactgcatgatccaggatctctcctggaaggtacgtacgcccgccccgccagagcagagcgccaagatcgatcgaccgaccgaccacacgtacgcgcctcgctcctgtcgctgaccgtggtttaatttgcgaaatgcgcagggccctgccaagaactgggagaacgtgctgctcagcctcggggtcgccggcggcgagccaggggtcgaaggcgaggagatcgcgccgctcgccaaggagaacgtggccgcgccctgaagagttcggcctgcaggccccctgatctcgcgcgtggtgcaaacatgttgggacatcttcttatatatgctgtttcgtttatgtgatatggacaagtatgtgtagctgcttgcttgtgctagtgtaatatagtgtagtggtggccagtggcacaacctaataagcgcatgaactaattgcttgcgtgtgtagttaagtaccgatcggtaattttatattgcgagtaaataaatggacctgtagtggtggagtaaataatccctgctgttcggtgttcttatcgcttctcgtatagatgttatatagagtacattttctttctgaatcctacgtttttcaagtgtttttgaattaa

SEQ ID NO: 15 - DNA, 27 bp, Zea mays
  source 1..27: mol_type = genomic DNA
tttcatccattggaacacacgcaactg

SEQ ID NO: 16 - DNA, 27 bp, Zea mays
  source 1..27: mol_type = genomic DNA
tttatgatcccgtgaagcctattttta

SEQ ID NO: 17 - DNA, 362 bp, Zea mays
  source 1..362: mol_type = genomic DNA
gcgtaccccgccgcgccgcacacgagcgcggccttcctcgctagcttcgcgacgccgctccccgcgcgccgcgcctcgcccccgacccggcgccgcgcggtgtcggtcgccgccatcgccaccaccaccacctgcgtcttcgccgacgccccccgtgggatcaaggaggagcaggcggactcgggcgcccaggcggcggcggctgcggctgcgcggagggacgtcagcccggacaccgtggcctccatcatcctcggcggtggcgccggcacgcgcctcttcccgctcacgcgtacaagggccaagcccgcggtgcccgtcgggggatgctacaggctcatcgacatcccgatgagcaactg

SEQ ID NO: 18 - DNA, 27 bp, Zea mays
  source 1..27: mol_type = genomic DNA
tttcagcaggatcctccggacaatttc

SEQ ID NO: 19 - DNA, 27 bp, Zea mays
  source 1..27: mol_type = genomic DNA
tttccccactccatgttgattacctgt

SEQ ID NO: 20 - DNA, 300 bp, Zea mays
  source 1..300: mol_type = genomic DNA
ggatgaacatggtctgtttaatgcactggaccttggcgggaccaactttcgtgtcatccgggtccaacttggcggaagggacagacgtgtcgtgaagctacagtatgaagaggtctccattccgcctcatcttatggttggaacttctacggaactatttgatttcattgctgctgagttggaacaatttgtgcggactgaaggagaagatttccacctaccagatggcaagcagagggaactgggtttcaccttttctttcccagtgcaccaaccatctatatcatcggggactctaat

SEQ ID NO: 21 - DNA, 27 bp, Zea mays
  source 1..27: mol_type = genomic DNA
tttggggaaagaccgaggagaagatct

SEQ ID NO: 22 - DNA, 27 bp, Zea mays
  source 1..27: mol_type = genomic DNA
tttaatttgcgaaatgcgcagggccct

SEQ ID NO: 23 - DNA, 330 bp, Zea mays
  source 1..330: mol_type = genomic DNA
ccctgcgcctgcgcgtccaccggtggactcgtcgacaccatcatcgaaggcaagaccgggttccacatcggccgcctcagcgtcgacgtaacgtcgtggagccggcggacgtcaagaaggtggccaccacattgcagcgcgccttcaaggtggtcggcacgccggcgtacgaggagatggtgaggaactgcttgatccaggatctctcctggaagggccctgccaagaactgggagaacgtgctgctcagcctcggggtcgccggcggctagccaggggtcgaaggcgaggagatcgcgccgctcgccaaggagaacgtggccgcgccct

SEQ ID NO: 24 - DNA, 23 bp, Zea mays
  source 1..23: mol_type = genomic DNA
acgactactacgcgctggggagg

SEQ ID NO: 25 - DNA, 23 bp, Zea mays
  source 1..23: mol_type = genomic DNA
gccctcgtacttggtccagttgg

SEQ ID NO: 26 - DNA, 250 bp, Zea mays
  source 1..250: mol_type = genomic DNA
tcgcgcgtctaccccaccgaggccatctacgccaacgccggcgtcttcctcttcaacaacgccaccgccgcgcgggtcacggccacgaagctcgtcgtccacgagatggactcgtcatacaaccacgactacatggcgccggtggcagacatctgatcgatgcaatgcaaccacgccactctgcaccgcaacaaattttccaacacacataaatataccaagcaattcccatctgtcaaattttcttatt

SEQ ID NO: 27 - DNA, 27 bp, Zea mays
  source 1..27: mol_type = genomic DNA
tttacttggtctgcaggaagggcattg

SEQ ID NO: 28 - DNA, 27 bp, Zea mays
  source 1..27: mol_type = genomic DNA
tttgtccaatcaacattcaatgccctt

SEQ ID NO: 29 - DNA, 23 bp, synthetic construct
  source 1..23: mol_type = other DNA
gacaaagctggatgacctttcat

SEQ ID NO: 30 - DNA, 24 bp, synthetic construct
  source 1..24: mol_type = other DNA
gttttgcagaagcatgttgactct

SEQ ID NO: 31 - DNA, 15 bp, synthetic construct
  source 1..15: mol_type = other DNA
acgcaactgaaatat

SEQ ID NO: 32 - DNA, 24 bp, synthetic construct
  source 1..24: mol_type = other DNA
accttgcagttttctaccttggtt

SEQ ID NO: 33 - DNA, 30 bp, synthetic construct
  source 1..30: mol_type = other DNA
tgtagtctcccaacttctatttttatgatc

SEQ ID NO: 34 - DNA, 20 bp, synthetic construct
  source 1..20: mol_type = other DNA
atgtaaaaataggcttcacg

SEQ ID NO: 35 - DNA, 29 bp, synthetic construct
  source 1..29: mol_type = other DNA
cagatatatgaaaagatgatttctggaat

SEQ ID NO: 36 - DNA, 21 bp, synthetic construct
  source 1..21: mol_type = other DNA
catcatgagccagtttcagca

SEQ ID NO: 37 - DNA, 16 bp, synthetic construct
  source 1..16: mol_type = other DNA
cttggagaaattgtcc

SEQ ID NO: 38 - DNA, 33 bp, synthetic construct
  source 1..33: mol_type = other DNA
ttctgtaattagtatcttaatatttctcaccag

SEQ ID NO: 39 - DNA, 22 bp, synthetic construct
  source 1..22: mol_type = other DNA
atctgaagtttccccactccat

SEQ ID NO: 40 - DNA, 17 bp, synthetic construct
  source 1..17: mol_type = other DNA
atgctacaggtaatcaa

SEQ ID NO: 41 - DNA, 20 bp, synthetic construct
  source 1..20: mol_type = other DNA
tgtagtccgttccagcgaca

SEQ ID NO: 42 - DNA, 21 bp, synthetic construct
  source 1..21: mol_type = other DNA
ggtttcaggtttggggaaaga

SEQ ID NO: 43 - DNA, 17 bp, synthetic construct
  source 1..17: mol_type = other DNA
ccgtagatcttctcctc

SEQ ID NO: 44 - DNA, 20 bp, synthetic construct
  source 1..20: mol_type = other DNA
gaggctgagcagcacgttct

SEQ ID NO: 45 - DNA, 21 bp, synthetic construct
  source 1..21: mol_type = other DNA
tgaccgtggtttaatttgcga

SEQ ID NO: 46 - DNA, 14 bp, synthetic construct
  source 1..14: mol_type = other DNA
ttggcagggccctg

SEQ ID NO: 47 - DNA, 26 bp, synthetic construct
  source 1..26: mol_type = other DNA
tcattatctgcagaactttgtccagt

SEQ ID NO: 48 - DNA, 30 bp, synthetic construct
  source 1..30: mol_type = other DNA
tgataagtaggttaccttttacttggtctg

SEQ ID NO: 49 - DNA, 16 bp, synthetic construct
  source 1..16: mol_type = other DNA
cattcaatgcccttcc

SEQ ID NO: 50 - DNA, 25 bp, synthetic construct
  source 1..25: mol_type = other DNA
ttatctgcagaactttgtccaatca

SEQ ID NO: 51 - DNA, 26 bp, synthetic construct
  source 1..26: mol_type = other DNA
aagcatgttacctcttacttgatcca

SEQ ID NO: 52 - DNA, 16 bp, synthetic construct
  source 1..16: mol_type = other DNA
cattcaatgcccttcc

SEQ ID NO: 53 - DNA, 16 bp, synthetic construct
  source 1..16: mol_type = other DNA
acgacgaccgccacga

SEQ ID NO: 54 - DNA, 15 bp, synthetic construct
  source 1..15: mol_type = other DNA
cggcgtccaggcgtt

SEQ ID NO: 55 - DNA, 15 bp, synthetic construct
  source 1..15: mol_type = other DNA
ctccccagcgcgtag

SEQ ID NO: 56 - DNA, 15 bp, synthetic construct
  source 1..15: mol_type = other DNA
gccgacccgctgctc

SEQ ID NO: 57 - DNA, 18 bp, synthetic construct
  source 1..18: mol_type = other DNA
cgggtacagcaccgggtt

SEQ ID NO: 58 - DNA, 16 bp, synthetic construct
  source 1..16: mol_type = other DNA
caactggaccaagtac

(19) United States
(12) Patent Application Publication
(10) Pub. No.: US 2026/0327639 A1
(43) Pub. Date: Oct. 22, 2026

(54) OBTAINING HAPLOIDS VIA ANDROGENESIS

(71) Applicant: Syngenta Crop Protection AG, Basel (CH)

(72) Inventors: Liu Weiguo, RTP, NC, NC (US); Xia Zhang, RTP, NC (US); Samson Prabhakar Nalapalli, Cary (YS); Julie Leonard Green, Woodland, CA (US); Gu Weining, RTP, NC (US); Zhongying Chen, RTP, NC (US); Chunyang Fan, RTP, NC (US)

(73) Assignee: Syngenta Crop Protection AG, Basel (CH)

(21) Appl. No.: 19/113,969

(22) PCT Filed: Sep. 18, 2023
(86) PCT No.: PCT/US2023/074445

§ 371 Date: Mar. 21, 2025

Related U.S. Application Data
(60) Provisional application No. 63/376,465, filed on Sep. 21, 2022.

(51) Int. Cl. C12N 15/82; C12N 9/22; C12N 15/10; C12N 15/113

(52) U.S. Cl. CPC C12N 15/8245; C12N 9/226; C12N 15/102; C12N 15/1137; C12N 15/8218; C12N 2310/14; C12N 2310/20

(57) ABSTRACT

Provided herein are methods for obtaining androgenic microspores. Also provided are methods for generating haploid plants from androgenic microspores. The methods include reducing expression of at least one pollen starch biosynthesis pathway gene. The methods also include androgenic microspores that have an increased response compared to a wildtype microspore. Further, the methods include reducing the expression of at least one pollen starch biosynthesis gene through a knockout or knockdown using gene editing or RNAi.

### FIELD OF THE INVENTION

The present invention generally relates to the production of doubled haploids through androgenesis.

### SEQUENCE LISTING

This application is accompanied by a sequence listing entitled 82745_ST26.xml, created Sep. 19, 2022 which is approximately 225 kilobytes in size. This sequence listing is incorporated herein by reference in its entirety. This sequence listing is submitted herewith via EFS-Web, and is in compliance with 37 C.F.R. § 1.824(a)(2)-(6) and (b).

### BACKGROUND

Microspores (“MS”) are haploid cells containing the gametic number of chromosomes and can be induced to form embryoids (pseudoembryos). This process is commonly referred to as microspore embryogenesis or androgenesis. Haploid embryoids produced from androgenesis can then develop into haploid plants. Haploid plants are generally frail and infertile. Chromosome doubling resulting from spontaneous or chemical induction doubles the chromosomes, resulting in a stable, homozygous, and fertile diploid or amphidiploid plants. Thus, microspores can form homozygous doubled haploid plants (DH) in one generation by androgenesis.

Millions of microspores can be easily isolated from one tassel of corn or a few spikes of wheat with a blender in a few minutes. In contrast, it takes much more effort to prepare similar numbers of immature haploid embryos, which requires a haploid inducer for haploid production followed by identification, isolation, and preparation of haploid embryos (“haploid induction”). Haploid induction generally produces fewer than 100 haploid embryos per induced ear. Thus, it is simpler and more economically beneficial to use microspores as the originating donor to obtain DH plants. Therefore, androgenesis, from haploid microspores to DH lines in one generation, is the most efficient DH production method. However, commercial plant breeding lacks a successful maize androgenesis method for a wide range of genotypes.

Previous androgenesis protocols called for applying treatments to microspores after their isolation. There is a correlation between chemical inducers and lack of starch formation in microspores and effects of androgenesis induction (Liu et al. Crop Science Volume 42, Issue 3. Pp. 686-692, 2002). Several genes have been found to be downregulated in microspores following mannitol treatment to induce barley androgenesis. Said down-regulation has also been shown to be analogous to the induction of a maltase gene and an invertase gene. Maltase and invertase are involved in starch and sucrose breakdown, respectively (Maraschin et al., Journal of Experimental Botany, Volume 56, Issue 417, July 2005, Pages 1711-1726). Thus, genes and their products in the starch accumulation pathway (Pfister & Zeeman, Cell. Mol. Life Sci. 73, 2781-2807 (2016).) in pollen are appropriate candidates as possible genes and/or signals that control triggering of androgenesis.

Blocking or interfering with the starch accumulation pathway and/or the pollen starch biosynthesis pathway can serve as the universal trigger of androgenesis by redirecting microspores from the pre-programmed gametophytic to the sporophytic development pathway. The present disclosure targets selected genes to block or interfere with the starch accumulation pathway in microspores that can serve as the universal trigger for androgenesis.

### SUMMARY

Hybrid breeding requires rapid development of pure inbreds. Current doubled haploid (DH) technology and methods are expensive. It takes great effort to prepare millions of immature haploid embryos through a standard haploid induction process. However, millions of microspores can be easily isolated from one tassel of corn (or a few spikes of wheat) with a blender in a few minutes. Thus, it is simpler and more economically beneficial to use microspores as the originating donor to obtain DH plants. Androgenesis, from haploid microspores to DH lines in one generation, is the most efficient DH production method. Currently, commercial plant breeding lacks a successful maize androgenesis method for a wide range of genotypes. Provided herein are methods to produce DH lines from microspores in one generation.

The methods herein reduced expression of at least one pollen starch biosynthesis pathway gene selected from ADP glucose pyrophosphorylase, Waxy1, Hexokinase5, Phosphoglucomutase1, Phosphoglucomutase2, and Invertase2. In these methods, the at least one pollen starch pathway gene is reduced by knockout or knockdown utilizing gene editing or RNAi. The gene editing is through the use of a site directed nuclease. For example, the gene editing is done using a CRISPR nuclease (e.g., Cas12a). In these methods, the androgenic response is increased at least 40% compared to the wildtype.

### BRIEF DESCRIPTION OF THE SEQUENCES IN THE SEQUENCE LISTING

SEQ ID NO: 1 is the nucleotide sequence for construct 25639.

SEQ ID NO: 2 is the nucleotide sequence for construct 25635.

SEQ ID NO: 3 is the nucleotide sequence for construct 25637.

SEQ ID NO: 4 is the nucleotide sequence for construct 25619.

SEQ ID NO: 5 is the nucleotide sequence for construct 25634.

SEQ ID NO: 6 is the nucleotide sequence for construct 25636.

SEQ ID NO: 7 is the nucleotide sequence for construct 25662.

SEQ ID NO: 8 is the nucleotide sequence for construct 25638.

SEQ ID NO: 9 is the nucleotide sequence for target gene phosphoglucomutase1.

SEQ ID NO: 10 is the nucleotide sequence for target gene phosphoglucomutase2.

SEQ ID NO: 11 is the nucleotide sequence for target gene ADP glucose pyrophosphorylaseII.

SEQ ID NO: 12 is the nucleotide sequence for target gene Invertase2.

SEQ ID NO: 13 is the nucleotide sequence for target gene Hexokinase5.

SEQ ID NO: 14 is the nucleotide sequence for target gene Waxy1.

SEQ ID NO: 15 is the nucleotide sequence for the gRNA target 1 of ADP glucose pyrophosphorylaseII.

SEQ ID NO: 16 is the nucleotide sequence for the gRNA target 2 of ADP glucose pyrophosphorylaseII.

SEQ ID NO: 17 is the nucleotide sequence for the RNAi target of ADP glucose pyrophosphorylaseII.

SEQ ID NO: 18 is the nucleotide sequence for the gRNA target 1 of Hexokinase5.

SEQ ID NO: 19 is the nucleotide sequence for the gRNA target 2 of Hexokinase5.

SEQ ID NO: 20 is the nucleotide sequence for the RNAi target of Hexokinase5.

SEQ ID NO: 21 is the nucleotide sequence for the gRNA target 1 of Waxy1.

SEQ ID NO: 22 is the nucleotide sequence for the gRNA target 2 of Waxy1.

SEQ ID NO: 23 is the nucleotide sequence for the RNAi target of Waxy1.

SEQ ID NO: 24 is the nucleotide sequence for the gRNA target 1 of Invertase2.

SEQ ID NO: 25 is the nucleotide sequence for the gRNA target 2 of Invertase2.

SEQ ID NO: 26 is the nucleotide sequence for the RNAi target of Invertase2.

SEQ ID NO: 27 is the nucleotide sequence for the gRNA target 1 of phosphoglucomutase1.

SEQ ID NO: 28 is the nucleotide sequence for the gRNA target 1 of phosphoglucomutase2.

SEQ ID NO: 29 is a primer sequence for TaqMan Assay 3682.

SEQ ID NO: 30 is a primer sequence for TaqMan Assay 3682.

SEQ ID NO: 31 is the probe sequence for TaqMan Assay 3682.

SEQ ID NO: 32 is a primer sequence for TaqMan Assay 3683.

SEQ ID NO: 33 is a primer sequence for TaqMan Assay 3683.

SEQ ID NO: 34 is the probe sequence for TaqMan Assay 3683.

SEQ ID NO: 35 is a primer sequence for TaqMan Assay 3684.

SEQ ID NO: 36 is a primer sequence for TaqMan Assay 3684.

SEQ ID NO: 37 is the probe sequence for TaqMan Assay 3684.

SEQ ID NO: 38 is a primer sequence for TaqMan Assay 3685.

SEQ ID NO: 39 is a primer sequence for TaqMan Assay 3685.

SEQ ID NO: 40 is the probe sequence for TaqMan Assay 3685.

SEQ ID NO: 41 is a primer sequence for TaqMan Assay 3686.

SEQ ID NO: 42 is a primer sequence for TaqMan Assay 3686.

SEQ ID NO: 43 is the probe sequence for TaqMan Assay 3686.

SEQ ID NO: 44 is a primer sequence for TaqMan Assay 3687.

SEQ ID NO: 45 is a primer sequence for TaqMan Assay 3687.

SEQ ID NO: 46 is the probe sequence for TaqMan Assay 3687.

SEQ ID NO: 47 is a primer sequence for TaqMan Assay 3688.

SEQ ID NO: 48 is a primer sequence for TaqMan Assay 3688.

SEQ ID NO: 49 is the probe sequence for TaqMan Assay 3688.

SEQ ID NO: 50 is a primer sequence for TaqMan Assay 3689.

SEQ ID NO: 51 is a primer sequence for TaqMan Assay 3689.

SEQ ID NO: 52 is the probe sequence for TaqMan Assay 3689.

SEQ ID NO: 53 is a primer sequence for TaqMan Assay 3690.

SEQ ID NO: 54 is a primer sequence for TaqMan Assay 3690.

SEQ ID NO: 55 is the probe sequence for TaqMan Assay 3690.

SEQ ID NO: 56 is a primer sequence for TaqMan Assay 3691.

SEQ ID NO: 57 is a primer sequence for TaqMan Assay 3691.

SEQ ID NO: 58 is the probe sequence for TaqMan Assay 3691.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the discloses compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “allele” refers to a variant or an alternative sequence form at a genetic locus. In diploids, a single allele is inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the term “amplified” or “amplify” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. When the phrase “consists of” appears in a clause of the body of a claim, rather than immediately following the preamble, it limits only the element set forth in that clause; other elements are not excluded from the claim as a whole.

With respect to the terms “comprising,” “consisting essentially of,” and “consisting of,” where one of these three terms is used herein, the presently disclosed and claimed subject matter can include in some embodiments the use of either of the other two terms. For example, if a subject matter relates in some embodiments to nucleic acids that encode polypeptides comprising amino acid sequences that are at least 95% identical to a SEQ ID NO: 2 or 3. It is understood that the disclosed subject matter thus also encompasses nucleic acids that encode polypeptides that in some embodiments consist essentially of amino acid sequences that are at least 95% identical to that SEQ ID NO: 2 or 3 as well as nucleic acids that encode polypeptides that in some embodiments consist of amino acid sequences that are at least 95% identical to that SEQ ID NO: 2 or 3. Similarly, it is also understood that in some embodiments the methods for the disclosed subject matter comprise the steps that are disclosed herein, in some embodiments the methods for the presently disclosed subject matter consist essentially of the steps that are disclosed, and in some embodiments the methods for the presently disclosed subject matter consist of the steps that are disclosed herein.

As used herein, the term “de novo haploid induction” refers to the triggering of haploid induction by the introduction of a spontaneous-haploid inducing agent. Such introduction can be achieved by topical spray, hand-pollination, mutagenesis, or transgenic methods. The terms “de novo haploid induction,” “de novo HI,” and “haploid induction de novo” are used interchangeably throughout this specification.

As used herein, the term “elite line” or “inbred line” refers to any line that has resulted from breeding and selection for superior agronomic performance. An elite line has stable genetics, i.e., it is reasonably or nearly isogenic across its genome. Said another way, an elite line is reasonably or nearly homozygous for all alleles in its genome.

As used herein, “embryoids” or “pseudoembryos” refers to clusters of embryo-like structures formed from a single microspore cell in vitro. These embryoids can form into fully grown plants. As used herein, “embryoid-like structures” or “ELS” refers to either embryos or callus that are formed from a single microspore cell and released out of the exine. “Callus” or “calli” refers to a mass of cells derived from microspore cell culture in vitro.

As used herein, the term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

As used herein, the term “gene” refers to a hereditary unit including a sequence of DNA that occupies a specific location on a chromosome and that contains the genetic instruction for a particular characteristic or trait in an organism.

As used herein, the term “genotype” refers to the genetic constitution of a cell or organism. An individual's “genotype for a set of genetic markers” includes the specific alleles, for one or more genetic marker loci, present in the individual. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a sum of one or more alleles present within an individual at one or more genetic loci of a quantitative trait. In some embodiments, a genotype is expressed in terms of a haplotype (defined herein below).

As used herein, the term “germplasm” refers to the totality of the genotypes of a population or another group of individuals (e.g., a species). The term “germplasm” can also refer to plant material; e.g., a group of plants that act as a repository for various alleles. The phrase “adapted germplasm” refers to plant materials of proven genetic superiority; e.g., for a given environment or geo-graphical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

As used herein, a plant referred to as “haploid” has a single set (genome) of chromosomes and the reduced number of chromosomes (1n) in the haploid plant is equal to that of the gamete. As used herein, a plant referred to as “doubled haploid” is developed by doubling the haploid set of chromosomes (from 1n to 2n). A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric.

As used herein, the term “heterologous” when used in reference to a gene or nucleic acid refers to a gene encoding a factor that is not in its natural environment (i.e., has been altered by the hand of man). For example, a heterologous gene may include a gene from one species introduced into another species. A heterologous gene may also include a gene native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the invention, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, in embodiments, a “heterologous” polynucleotide is a polynucleotide not naturally associated with a host cell into which it is introduced, including non-naturally occurring multiple copies of a naturally occurring polynucleotide.

As used herein, the term “heterozygous” means a genetic condition existing when different alleles reside at corresponding loci on homologous chromosomes.

As used herein, the term “homozygous” means a genetic condition existing when identical alleles reside at corresponding loci on homologous chromosomes.

As used herein, the term “isolated,” when used in the context of the nucleic acid molecules or polynucleotides of the present invention, refers to a polynucleotide that is identified within and isolated/separated from its chromosomal polynucleotide context within the respective source organism. An isolated nucleic acid or polynucleotide is not a nucleic acid as it occurs in its natural context, if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The isolated nucleic acid molecule may be present in single-stranded or double-stranded form. Alternatively, it may contain both the sense and antisense strands (i.e., the nucleic acid molecule may be double-stranded). In a preferred embodiment, the nucleic acid molecules of the present invention are understood to be isolated.

As used herein, the term “locus” refers to a position (e.g., of a gene, a genetic marker, or the like) on a chromosome of a given species.

As used herein, the term “human-induced mutation” refers to any mutation that occurs as a result of either direct or indirect human action. This term includes, but is not limited to, mutations obtained by any method of targeted mutagenesis.

As used herein, the term “hybrid” refers to offspring produced by crossing two genetically dissimilar parent plants. The resulting progeny of this cross are a “bi-parental” population.

As used herein, the terms “marker probe” and “probe” refer to a nucleotide sequence or nucleic acid molecule that can be used to detect the presence or absence of a sequence within a larger sequence, e.g., a nucleic acid probe that is complementary to all of or a portion of the marker or marker locus, through nucleic acid hybridization. Marker probes comprising about 8, 10, 15, 20, 30, 40, 50, 60, 70, 80, 90, 100 or more contiguous nucleotides can be used for nucleic acid hybridization.

As used herein, the term “molecular marker” can be used to refer to a genetic marker, as defined above, or an encoded product thereof (e.g., a protein) used as a point of reference when identifying the presence/absence of a SCD or other locus of interest. A molecular marker can be derived from genomic nucleotide sequences or from expressed nucleotide sequences (e.g., from an RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution (e.g., according to Watson-Crick base pairing rules). This term also refers to the genetic markers that indicate a trait by the absence of the nucleotide sequences complementary to or flanking the marker sequences, such as nucleotide sequences used as probes and/or primers capable of amplifying the marker sequence.

As used herein, the terms “nucleotide sequence,” “polynucleotide,” “nucleic acid sequence,” “nucleic acid molecule,” and “nucleic acid fragment” refer to a polymer of RNA or DNA that is single- or double-stranded, optionally containing synthetic, non-natural, and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/and as described in the following publication: “The EMBL-EBI search and sequence analysis tools APIs in 2019.” Madeira et al. Nucleic Acids Research, June 2019, 47(W1):W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiments, substantially identical nucleic acid or amino acid sequences may perform substantially the same function.

As used herein, the terms “introgression”, “introgressed” and “introgressing” refer to both a natural and artificial process whereby genomic regions of one species, variety or cultivar are moved into the genome of another species, variety or cultivar, by crossing those species. The process may optionally be completed by backcrossing to the recurrent parent.

The term “open reading frame” (ORF) refers to a nucleic acid sequence that encodes a polypeptide. In some embodiments, an ORF comprises a translation initiation codon, a translation termination (i.e., stop) codon, and the nucleic acid sequence there between that encodes the amino acids present in the polypeptide. The terms “initiation codon” and “termination codon” refer to a unit of three adjacent nucleotides (i.e., a codon) in a coding sequence that specifies initiation and chain termination, respectively, of protein synthesis (mRNA translation).

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). For example, in the case of a haploid plant evaluated for SCD, phenotype can refer to fertile pollen shed and/or a seed obtained via pollination with that pollen. In other cases, a phenotype is the result of interactions among several genes, which in some embodiments also results from an interaction of the plant and/or plant cell with its environment.

As used herein, the term “plant” can refer to a whole plant, any part thereof, or a cell or tissue culture derived from a plant. Thus, the term “plant” can refer to any of: whole plants, plant components or organs (e.g., leaves, stems, roots, etc.), plant tissues, seeds and/or plant cells, unless otherwise specified.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “population” means a genetically heterogeneous collection of plants sharing a common genetic derivation.

As used herein, the term “primer” refers to an oligonucleotide which is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

As used herein, the terms “progeny” and “progeny plant” refer to a plant generated from a vegetative or sexual reproduction from one or more parent plants. In haploid induction the seed on the female parent is haploid, thus not a progeny of the inducing haploid line. The progeny of the haploid seed is not the only desired progeny. There is also the HI seed and subsequent plant and seed progeny of the haploid inducing plant. Both the haploid seed and the HI seed can be progeny. A progeny plant can be obtained by cloning or selfing a single parent plant, or by crossing two or more parental plants. For instance, a progeny plant can be obtained by cloning or selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1s, F2s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids.

As used herein, the term “regenerate,” and grammatical variants thereof, refers to the production of a plant from tissue culture.

As used herein, “spontaneously doubled haploid plant” refers to a plant whose florets have undergone spontaneous doubling. Other tissues of a spontaneously doubled haploid plant may retain their haploid state (e.g., root, leaf, stem).

As used herein, the term “trait” refers to a phenotype of interest, a gene that contributes to a phenotype of interest, as well as a nucleic acid sequence associated with a gene that contributes to a phenotype of interest.

As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome.

As used herein, haploid induction rate (“HIR”) means the number of surviving haploid kernels over the total number of kernels after an ear is pollinated with haploid inducer pollen.

### DETAILED DESCRIPTION

Described herein is a method of obtaining an androgenic microspore. The method comprises reducing expression of at least one pollen starch biosynthesis pathway gene, wherein the androgenic microspore has an increased androgenic response compared to a wildtype microspore. The method further comprises that the expression of at least one pollen starch biosynthesis pathway gene is reduced by knockout or knockdown. In an embodiment, the pollen starch biosynthesis pathway gene is selected from the group consisting of ADP glucose pyrophosphorylase, Waxy1, Hexokinase5, Phosphoglucomutase1 and Phosphoglucomutase2, and Invertase2. In another embodiment, the ADP glucose pyrophosphorylase, Waxy1, Hexokinase5, Phosphoglucomutase1 and Phosphoglucomutase2, and Invertase2 genes comprise SEQ ID NO: 11, SEQ ID NO: 14, SEQ ID NO: 13, SEQ ID NO: 9, SEQ ID NO: 10, and SEQ ID NO: 12, respectively.

The pollen starch biosynthesis pathway gene knockout may be achieved through gene editing while the knockdown may be achieved through RNAi. In an embodiment, the gene editing is done using a site directed nuclease selected from the group consisting of a CRISPR nuclease, a meganuclease, a zinc-finger nuclease, and a transcription-activator like effector nuclease. In one embodiment, the site-directed nuclease is a CRISPR nuclease, and in another embodiment, the CRISPR nuclease is selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof. In a further embodiment, the CRISPR nuclease is Cas12a. In one embodiment, the knockout is a single gene knockout comprising use of two gRNAs and the two gRNAs comprise SEQ ID NOs: 15 and 16. In one embodiment, the knockout is a single gene knockout comprising use of two gRNAs and the two gRNAs comprise SEQ ID NOs: 18 and 19. In one embodiment, the knockout is a single gene knockout comprising use of two gRNAs and the two gRNAs comprise SEQ ID NOs: 21 and 22. In one embodiment, the knockout is a single gene knockout comprising use of two gRNAs and the two gRNAs comprise SEQ ID NOs: 24 and 25. In one embodiment, the knockout comprises use of multiplexed gRNAs, wherein the gRNAs comprise SEQ ID NOs: 15, 16, 18, 19, 21, 22, 27, and 28. In one embodiment, the increased androgenic response is increased at least 40% compared to the wild type. In another embodiment, obtained microspores are selected from the group consisting of maize, rice, and wheat. The obtained microspores may be from maize.

Also described herein are methods of generating a haploid plant. The methods comprise of first obtaining androgenic microspores, wherein the androgenic microspores have an increased androgenic response compared to the wild type. The obtained microspores are cultured, and a haploid plant is regenerated from said microspores. In an embodiment, the increased androgenic response is achieved through knocking out or knocking down at least one pollen starch biosynthesis pathway gene. The pollen starch biosynthesis pathway gene is selected from the group consisting of ADP glucose pyrophosphorylase, Waxy1, Hexokinase5, Phosphogluconmutase1 and Phosphoglucomutase2, and Invertase2 and may comprise SEQ ID NOs: 11, SEQ ID NO: 14, SEQ ID NO: 13, SEQ ID NO: 9, SEQ ID NO: 10, and SEQ ID NO: 12, respectively.

In one embodiment, the knockout may be achieved through gene editing while the knockdown may be achieved through RNAi. The gene editing may be done using a site directed nuclease selected from the group consisting of a CRISPR nuclease, a meganuclease, a zinc-finger nuclease, and a transcription-activator like effector nuclease. In one embodiment, the site directed nuclease is a CRISPR nuclease and CRISPR nuclease may be selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof. In one embodiment, the CRISPR nuclease may be Cas12a.

In one embodiment, the knockout is a single gene knockout comprising use of two gRNAs and the two gRNAs comprise SEQ ID NOs: 15 and 16. In one embodiment, the knockout is a single gene knockout comprising use of two gRNAs and the two gRNAs comprise SEQ ID NOs: 18 and 19. In one embodiment, the knockout is a single gene knockout comprising use of two gRNAs and the two gRNAs comprise SEQ ID NOs: 21 and 22. In one embodiment, the knockout is a single gene knockout comprising use of two gRNAs and the two gRNAs comprise SEQ ID NOs: 24 and 25. In one embodiment, the knockout comprises use of multiplexed gRNAs, wherein the gRNAs comprise SEQ ID NOs: 15, 16, 18, 19, 21, 22, 27, and 28. In one embodiment, the increased androgenic response is increased at least 40% compared to the wild type. In another embodiment, obtained microspores are selected from the group consisting of maize, rice, and wheat. The obtained microspores may be from maize.

In another embodiment, the methods comprise doubling the chromosomes of the haploid plant to produce a doubled haploid plant. The chromosome doubling may be achieved via chemical induction or spontaneous doubling. In an embodiment, chromosome doubling is achieved via chemical induction and achieved by a chemical selected from the group consisting of colchicine, trifluralin, pronamide, dithipyr, nitrous oxide, and oryzalin. In an embodiment, the method of generating doubled haploid plants from microspores occurs in one generation. Also provided herein are plants produced from the methods described.

### Examples

### 1. Androgenesis Protocol for Testing Present Hypothesis

A modified androgenesis protocol (See Zheng et al., 2003) was used to compare GE-modified microspores with wild type microspores for potential androgenesis (i.e., number of embryoid-like structures from 100 spikelets). Tassels with microspores at mid-to-late uninucleate stage were harvested, wrapped in aluminum foil, and stored in the dark at 6-8° C. for 14 days. Then, the microspores were isolated with a blender and purified with maltose gradient centrifugation. Isolated microspores were cultured at 28° C. in the dark with two fresh wheat ovaries per mL of culture media from wheat variety AC Nanda to generate calli or embryoids. This protocol was used as a baseline for comparing GE-modified microspores to non-modified microspores for androgenesis potential.

### 2. Transformable Genotypes, Candidate Gene Selection, Vector Construction, and Event Selection

### Genotypes

There were four genotypes (3 inbreds and 1 hybrid (F1)) tested to select options for later transformation to produce transgenic microspores. Hybrid 1 and Inbred 1 were selected for transformation to host the designed constructs based on their transformability data and preferences (Table 1). The inbred was selected solely for the FIREWORKS editing approach while the hybrid was selected for all eight constructs in Table 2 below.

TABLE 1
List of genotypes tested for transformability to host constructs.

| Experiment | | | Selectable | Valid | Total | Transformation |
|---|---|---|---|---|---|---|
| ID | Variety | Construct(s) | Marker | Explants | Events | Frequency |
| 1 | Inbred 2 | 12672 | PMI | 300 | 0 | 0 |
| 2 | Inbred 2 | 23967 | PMI | 300 | 4 | 1.33 |
| 3 | Inbred 1 | 12672 | PMI | 170 | 2 | 1.17* |
| 5 | Hybrid 1 | 12672 | PMI | 300 | 19 | 6.33** |
| N/A | Inbred 3 | N/A*** | N/A | N/A | N/A | N/A |

*Chosen for the FIREWORKS construct only
**Chosen for all 8 constructs
***Did not produce sufficient ears for enough immature embryos

### Gene Selection

Five genes in the maize pollen starch biosynthesis pathway were selected to demonstrate that disruption in the starch biosynthesis pathway leads to increased androgenesis: ADP glucose pyrophosphorylase, Waxy1, Hexokinase5, Phosphoglucomutase1 and Phosphoglucomutase2, and Invertase2. We generated T0 events using two strategies: knockout (loss of function) via CRISPR gene editing and knockdown (reduced expression) via RNAi.

### Vector Construction

We created three types of constructs for the two strategies mentioned above. First, we created single gene knockout constructs using two gRNAs per gene. Secondly, we created single gene knockdown constructs using RNAi. Finally, we used a FIREWORKS approach (See WO2020/176412) to multiplex gRNAs for multiple genes to produce microspores with a multi-gene knockout. Table 2 shows the various constructs generated and tested.

TABLE 2
Table 2 displays a list of the various constructs created and tested
with the pollen starch biosynthesis pathway gene targets.

| Target gene(s) | Construct Type | Construct number |
|---|---|---|
| Waxy1 | KD by RNAi | 25639 |
| Hexokinase 5 | KO by editing | 25635 |
| Hexokinase 5 | KD by RNAi | 25637 |
| ADP glucose | KO FIREWORKS | 25619 |
| pyrophosphorylase II, | | |
| Waxy1, Hexokinase5, | | |
| Phosphoglucomutase1, | | |
| Phosphoglucomutase 2 | | |
| ADP glucose | KO by editing | 25634 |
| pyrophosphorylase II | | |
| ADP glucose | KD by RNAi | 25636 |
| pyrophosphorylase II | | |
| Invertase2 | KO by editing | 25662 |
| Invertase2 | KD by RNAi | 25638 |

KD = knockdown;
KO = knockout

### Event Selection

We screened the T0 events and subsequent T1 plants for CRISPR/Cas12a induced gene editing and RNAi-mediated gene silencing using the following method. T0 plants were generated through Agrobacterium-mediated transformation following established procedures (Zhong et al., 2018). Genomic DNA and total RNA was isolated from the T0 plants and subjected to molecular analysis. A TaqMan copy number assay combined with targeted Next Generation Sequencing (NGS) or a TaqMan copy number assay combined with qRT-PCR was used to detect the knockout (KO) and knockdown (KD) events, respectively. TO transgenic plants carrying monoallelic or biallelic mutations as well as a single copy of the transgene without vector backbone were selected as T0 KO plants. Transgenic plants showing reduced gene expression by qRT-PCR were selected as T0 KD events. The selected T0 events were grown in a greenhouse and crossed with the DH inbred line 1 as the female to generate T1 plants (T0×inbred line 1). Androgenic response was evaluated on both T0 and T1 plants. Non-transgenic plants were used as a control.

### 3. Androgenesis Evaluation on Microspores

In our evaluation, we observed three instances of increased androgenic response of GE-microspores of KO or KD genes at T0 generation as well as the subsequent T1 generation (produced by a first back cross). First, the most responsive KD T0 event was in the hybrid line 1 genotype from RNAi construct 25639. This event showed that repressed expression of Waxy1 resulted in over 300% increase of androgenic response in terms of the production of embryoid-like structures (ELS) from 100 spikelets compared to the non-edited microspores (wild type). The subsequent T1 microspores also had over a 300% increase of androgenic response. The T2 microspores had a 155% average increase of androgenic response in terms of the production of ELS from 200 thousand microspores.

Secondly, a KO construct (25635) targeting the Hexokinase 5 gene in hybrid line 1 resulted in a 70% increase of androgenic response in terms of ELS from cultured TO microspores from 100 spikelets compared to the non-edited microspores (wild type). The subsequent T1 generation microspores had a 338 to 602% increase of androgenic response (measured as ELS from two hundred thousand microspores). This increase in the T1 generation, compared to the T0 generation, may have been due to less stress and short plants as was the case in T0. T2 progeny from three different progenitor T1 plants showed a 575 to 1406% increase of androgenic response compared to non-edited microspores (measured as ELS from two hundred thousand microspores).

Finally, a FIREWORK construct (25619) simultaneously targeting several genes (ADP glucose pyrophosphorylase, Waxy1, Hexokinase 5, and Phosphoglucomutase) in inbred line 1 resulted in over a 400% increase of androgenic response in terms of ELS from cultured TO microspores from 100 spikelets compared to non-edited microspores (wild type). However, T1 seeds were not generated from the best responsive TO event.

TABLE 3
The table below shows androgenic results from all tested constructs in the T0 and T1 generations.
The three specific increased response instances discussed above in Example 3 are bold in font.

| | | Mean T0 androgenic | Mean T1 androgenic | |
|---|---|---|---|---|
| | | response vs control | response vs control | |
| Target Gene | Construct | (ELS from 100 spikelets) | (ELS from 200K MS) | Genotype |
| Waxy1 | 25639(KD) | 386% increase | 362% increase | Hybrid Line 1 |
| Hexokinase 5 | 25635(KO) | 70% increase | 338%-602% increase | Hybrid Line 1 |
| Hexokinase 5 | 25637 (KD) | 160% increase | NS | Hybrid Line 1 |
| ADP glucose | 25619 (KO | 464% increase | Seeds not generated | Inbred Line 1 |
| pyrophosphorylase II, | FIREWORKS) | | from best T0 event | |
| Waxy1, Hexokinase5, | | | | |
| Phosphoglucomutase1 | | | | |
| and 2 | | | | |
| ADP glucose | 25619 (KO | NS, but higher | Not tested | Hybrid Line 1 |
| pyrophosphorylase II, | FIREWORKS) | number of initial | | |
| Waxy1, Hexokinase5, | | cell division (multi- | | |
| Phosphoglucomutase 1 | | cell structures) | | |
| and 2 | | | | |
| ADP glucose | 25634 (KO) | NS | NS | Hybrid Line 1 |
| pyrophosphorylase II | | | | |
| ADP glucose | 25636 (KD) | 13% increase | NS | Hybrid Line 1 |
| pyrophosphorylase II | | | | |
| Invertase2 | 25662 (KO) | No KO event | No KO event | Hybrid Line 1 |
| Invertase2 | 25638 (KD) | No KD event | No KD event | Hybrid Line 1 |

KD = knockdown,
KO =knockout,
NS = not significant

TABLE 4
The table below shows androgenic results for the T2 generation
from the most responsive plants of the T1 generation from
Hybrid Line 1 (first two rows above in Table 3).

| | | Mean T2 androgenic response vs. |
|---|---|---|
| | | control (Embryoid-like structure |
| Target Gene | Construct | per 200K MS) |
| Waxy1 | 25639 (KD) | 155% avg. increase over 3 plants |
| Hexokinase 5 | 25635 (KO) | 575%-1406% increase over 20 plants |

### REFERENCES

1. Zheng, M. Y., Weng, Y., Sahibzada, R., Konzak, C. F. (2003). Isolated microspore culture in maize (Zea mays L.), production of doubled-haploids via induced androgenesis. In: Maluszynski, M., Kasha, K. J., Forster, B. P., Szarejko, I. (eds) Doubled Haploid Production in Crop Plants. Springer, Dordrecht. https://doi.org/10.1007/978-94-017-1293-4_15
2. Weiguo Liu, Ming Y. Zheng, Enrique A. Polle, Calvin F. Konzak. Highly Efficient Doubled-Haploid Production in Wheat (Triticum aestivum L.) via Induced Microspore Embryogenesis. 2002. Crop Science Volume 42, Issue 3. Pp. 686-692. https://doi.org/10.2135/cropsci2002.6860
3. S. F. Maraschin, W. de Priester, H. P. Spaink, M. Wang, Androgenic switch: an example of plant embryogenesis from the male gametophyte perspective. 2005. Journal of Experimental Botany, Volume 56, Issue 417, July 2005, Pages 1711-1726, https://doi.org/10.1093/jxb/eri190.
4. Pfister, B., Zeeman, S. C. Formation of starch in plant cells. Cell. Mol. Life Sci. 73,2781-2807 (2016). https://doi.org/10.1007/s00018-016-2250-x.
5. Zhong, H. et al. (2018). Advances in Agrobacterium-mediated Maize Transformation. In: Lagrimini, L. (eds) Maize. Methods in Molecular Biology, vol 1676. Humana Press, New York, NY. https://doi.org/10.1007/978-1-4939-7315-6_3.

What is claimed is:

1. A method of obtaining an androgenic microspore, comprising reducing expression of at least one pollen starch biosynthesis pathway gene, wherein the androgenic microspore has an increased androgenic response compared to a wildtype microspore.

2. The method of claim 1, wherein the expression of at least one pollen starch biosynthesis pathway gene is reduced by knock out through gene editing or knock down through RNAi.

3. (canceled)

4. (canceled)

5. The method of claim 2, wherein the gene editing is done using a site directed nuclease selected from the group consisting of a CRISPR nuclease, a meganuclease, a zinc-finger nuclease, and a transcription-activator like effector nuclease.

6. The method of claim 5, wherein the site-directed nuclease is a CRISPR nuclease, wherein the CRISPR nuclease is selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof.

7. (canceled)

8. The method of claim 6, wherein the CRISPR nuclease is Cas12a.

9. The method of claim 2, wherein the knockout is a single gene knockout comprising use of two gRNAs, wherein the two gRNAs are selected from the group consisting of two gRNAs comprising SEQ ID NOs: 15 and 16, two gRNAs comprising SEQ ID NOs: 18 and 196, two gRNAs comprising SEQ ID NOs: 21 and 22, and two gRNAs comprising SEQ ID NOs: 24 and 25.

10. (canceled)

11. (canceled)

12. (canceled)

13. (canceled)

14. The method of claim 2, wherein the knockout comprises use of multiplexed gRNAs, wherein the gRNAs comprises SEQ ID NOs: 15, 16, 18, 19, 21, 22, 27, and 28.

15. (canceled)

16. The method of claim 2, wherein the pollen starch biosynthesis pathway gene is selected from the group consisting of ADP glucose pyrophosphorylase, Waxy1, Hexokinase5, Phosphogluconmutase1, Phosphoglucomutase2, and Invertase2, wherein the ADP glucose pyrophosphorylase, Waxy1, Hexokinase5, Phosphoglucomutase, and Invertase2 genes comprise SEQ ID NO: 11, SEQ ID NO: 14, SEQ ID NO: 13, SEQ ID NO: 9, SEQ ID NO: 10, and SEQ ID NO: 12, respectively.

17. (canceled)

18. The method of claim 1, wherein the increased androgenic response is increased at least 40% compared to the wild type.

19. The method of claim 1, wherein the obtained microspores are selected from the group consisting of maize, rice, and wheat.

20. (canceled)

21. A method of generating a haploid plant comprising
a) obtaining androgenic microspores, wherein the androgenic microspores have an increased androgenic response compared to the wild type,
b) culturing the microspores, and
c) regenerating a haploid plant from the microspores of b).

22. The method of claim 21, wherein the increased androgenic response is achieved through knocking out through gene editing or knocking down through RNAi at least one pollen starch biosynthesis pathway gene.

23. (canceled)

24. (canceled)

25. The method of claim 2, wherein the gene editing is done using a site directed nuclease selected from the group consisting of a CRISPR nuclease, a meganuclease, a zinc-finger nuclease, and a transcription-activator like effector nuclease.

26. The method of claim 25, wherein the site directed nuclease is a CRISPR nuclease selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof.

27. (canceled)

28. (canceled)

29. The method of claim 22, wherein the knockout is a single gene knockout comprising use of two gRNAs, wherein the two gRNAs are selected from the group consisting of two gRNAs comprising SEQ ID NOs: 15 and 16, two gRNAs comprising SEQ ID NOs: 18 and 196, two gRNAs comprising SEQ ID NOs: 21 and 22, and two gRNAs comprising SEQ ID NOs: 24 and 25.

30. (canceled)

31. (canceled)

32. (canceled)

33. (canceled)

34. The method of claim 22, wherein the knockout comprises use of multiplexed gRNAs, wherein the gRNAs comprise SEQ ID NOs: 15, 16, 18, 19, 21, 22, 27 and 28.

35. (canceled)

36. The method of claim 21, wherein the pollen starch biosynthesis pathway gene is selected from the group consisting of ADP glucose pyrophosphorylase, Waxy1, Hexokinase5, Phosphoglucomutase, and Invertase2, wherein the ADP glucose pyrophosphorylase, Waxy1, Hexokinase5, Phosphoglucomutase1, Phosphoglutomutase2, and Invertase2 genes comprise SEQ ID NO: 11, SEQ ID NO: 14, SEQ ID NO: 13, SEQ ID NO: 9, SEQ ID NO: 10, and SEQ ID NO: 12, respectively.

37. (canceled)

38. The method of claim 21, wherein the androgenic response is increased at least 40% compared to the wild type.

39. The method of claim 21, wherein chromosomes of the haploid plant are doubled to produce a doubled haploid plant, wherein the chromosome doubling is achieved via chemical induction or spontaneous doubling.

40. (canceled)

41. The method of claim 39, wherein the chromosome doubling is achieved via chemical induction using a chemical selected from the group consisting of colchicine, trifluralin, pronamide, dithipyr, nitrous oxide, and oryzalin.

42. (canceled)

43. The method of claim 39, wherein the method of generating doubled haploid plants from microspores occurs in one generation.

44. The method of claim 21, wherein the obtained microspores are selected from the group consisting of maize, rice, and wheat.

45. (canceled)

46. A plant produced by the method of claim 21.

47. A plant produced by the method of claim 39.
